(19) United States
(12) Patent Application Publication
(10) Pub. No.: US 2004/0110727 A1
(43) Pub. Date: Jun. 10, 2004

(54) Aldehyde-releasing compounds

(76) Inventors: Donald R Phillips, Victoria (AU); Susanne M. Cutts, Victoria (AU); Ada Raphaell, Herzalia (IL); Abraham Nudelman, Rehovot (IL)

Correspondence Address: FOLEY AND LARDNER, SUITE 500, 3000 K STREET NW, WASHINGTON, DC, 20007, US

(21) Appl. No.: 10/398,208

(22) PCT Filed: Oct. 5, 2001
(86) PCT No.: PCT/AU01/01254

(30) Foreign Application Priority Data
Oct. 6, 2000 (AU) PR 0594

(51) Int. Cl. A61K 31/66; A61K 31/445; A61K 31/36

(52) U.S. Cl. 514/114; 514/316; 514/423; 514/464

(57) ABSTRACT

A method of treating cancer, comprising the step of administering to a subject in need thereof an effective amount of a compound or compounds which increase or supplement the intracellular levels of endogenous aldehyde, prior to, together with, or subsequent to the administration of a therapeutically-effective amount of a chemotherapeutic agent such as anthracyclines and anthracenediones, wherein the efficacy of the chemotherapeutic agent is enhanced relative to the efficacy of the chemotherapeutic agent alone. The compound may be an aldehyde-releasing compound (preferably formaldehyde), including known aldehyde-releasing compounds and two new classes of aldehyde-releasing compounds. One new class of aldehyde-releasing compounds of formula (II) release more than one equivalent of aldehyde Z-(L-M¹-CHR-M²)ₓ (II) wherein x is an integer of 2 or more Z is a direct bond or a linking group of valency x; L is either a direct bond or a spacer group R is H or C1-4 alkyl, alkenyl or alkynyl; M¹ is a decomposable or hydrolysable group; and M² is a second decomposable or hydrolysable group. Another new class of aldehyde-releasing compounds include a radical based on an inhibitor of an aldehyde detoxifying agent, such as buthionine sulphoximine or crotonaldehyde.

This invention relates to compounds, compositions and methods for potentiating the action of chemotherapeutic agents, for reducing the dose required for therapeutic effectiveness, and for overcoming resistance to the chemotherapeutic agent. The invention also provides novel aldehyde-releasing compounds which increase the efficacy of chemotherapeutic agents.

### BACKGROUND OF THE INVENTION

Many diseases that afflict animals, including humans, are treated with chemotherapeutic agents. For example, chemotherapeutic agents have proven valuable in the treatment of neoplastic disorders such as cancer, connective or autoimmune diseases, metabolic disorders, and dermatological diseases. Some of these agents are highly effective and do not suffer from any bioavailability or toxic side effect problems such as neutropenia. Unfortunately, many chemotherapeutic agents have severe problems with bioavailability and/or toxic side effects that adversely affect their clinical usefulness.

The anthracycline group of compounds contains some of the most widely used of all the anti-cancer agents in current clinical use, including Adriamycin which is used in the treatment of a wide range of tumours (Weiss, 1992; DeVita et al., 1993; Pratt et al., 1994; Bishop, 1999). In addition to Adriamycin, other members of this group including daunomycin (2), idarubicin (3), and epirubicin (4) are commonly used. The chemical, biochemical and pharmacological properties of these chemotherapeutic agents have been described in detail (Myers et al., 1988; DeVita et al., 1993; Sweatman and Israel, 1997; Gewirtz, 1999; Phillips and Cullinane, 1999).

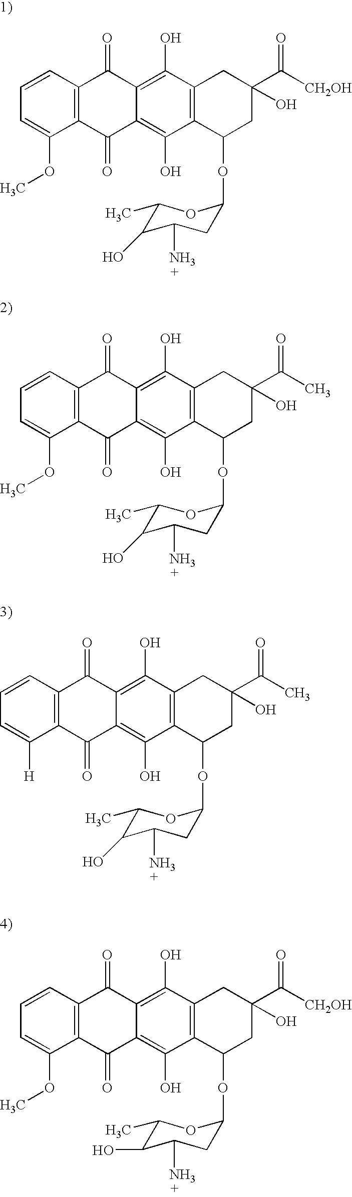

Structures of commonly used anthracyclines 1, Adriamycin; 2, daunomycin; 3, idarubicin; 4, epirubicin.

Although the anthracyclines have been used for well over two decades, their mechanism of action is not yet fully understood. The lack of understanding of the molecular details of the mechanism of action of the anthracyclines has hindered the development of improved anthracyclines, and this has been exemplified by the fact that over 2,000 derivatives have been assessed to date without yielding new derivatives with substantially improved activity (Weiss, 1992; Phillips and Cullinane, 1999).

The clinical use of the anthracyclines has also been hindered by two further problems:

1. a cumulative, dose-dependent cardiomyopathy which restricts the maximum recommended dose to 550 mg/m²;

2. the development of resistance following extended periods of use, due primarily to the over-expression of the active efflux pump P-glycoprotein, but also to a range of other detoxification mechanisms (Chabner and Myers, 1993; Pratt et al., 1994; Sweatman and Israel, 1997).

Using a transcription assay, the Applicant has shown that Adriamycin is able to form adducts with DNA under appropriate in vitro conditions, and that these adducts form almost exclusively at guanine residues, although mainly at 5′-GC-3′ sequences. The Applicant has also shown that these adducts can be detected in both the nuclear and mitochondrial DNA of cells in culture which have been exposed to Adriamycin. Moreover, the Applicant has revealed a clear correlation between the formation of these DNA adducts and a cytotoxic response, as well as a requirement for the presence of aldehyde, and in particular formaldehyde. The aldehyde has been found to be advantageously provided to the system by a compound that releases aldehyde in situ. Applicant has also found that there was a surprising dependence on the relative order and timing of addition of the chemotherapeutic agent/aldehyde-releasing compound combination.

From these results the Applicant has now defined a model for the molecular processes involved in the action of Adriamycin, and how the cellular responses to Adriamycin can be directed down one reaction pathway by the use of aldehyde-releasing compounds (also referred to as an. “aldehyde-releasing prodrug” or “prodrug”). This model is shown below:

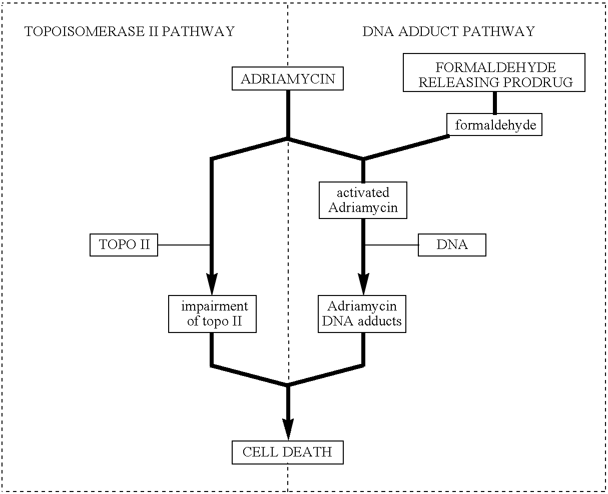

This model shows that Adriamycin and other anthracyclines may induce cell killing by several different mechanisms. For simplicity only two such possible pathways have been shown: one involving impairment of the topological enzyme topoisomerase II, the apparent central target, and the other involving the formation of DNA adducts. On the basis of this model the Applicant predicts that in the presence of excess aldehydes, in particular formaldehyde, Adriamycin will be directed down the DNA adduct pathway.

This leads to several predictions as to the cellular response under such conditions:

(1) The number of adducts will increase with the aldehyde-releasing compound:chemotherapeutic agent ratio, up to saturation of the available chemotherapeutic agent pool;

(2) The greater the number of DNA adducts, the greater will be the cytotoxic response; and

(3) The time of addition of chemotherapeutic agent and aldehyde-releasing compound will affect the extent of adduct formation.

As shown herein, all of these responses have been confirmed in cells treated in culture with Adriamycin and the aldehyde-releasing compound AN-9, providing good evidence in support of the model.

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

For the purposes of this specification it will be clearly understood that the word “comprising” means “including but not limited to”, and that the word “comprises has a corresponding meaning.

### SUMMARY OF THE INVENTION

Applicant has found that certain chemotherapeutic agents such as anthracyclines and related compounds such as anthracenediones, when combined with compounds that increase or supplement the intracellular levels of aldehyde, such as aldehyde-releasing compounds, result in enhanced levels of formation of drug-DNA adducts, leading to an increased cytotoxic response, the response being defined by the relative aldehyde-releasing compound:chemotherapeutic agent ratio, relative time and duration of administration. This could enable decreased levels of the chemotherapeutic agent to be used, thereby reducing the risk of toxic side effects of the therapy.

Accordingly, in a first aspect, the invention provides a method of treating cancer, comprising the step of administering to a subject in need thereof an effective amount of a compound or compounds which increase or supplement the intracellular levels of endogenous aldehyde, prior to, together with, or subsequent to the administration of a therapeutically-effective amount of a chemotherapeutic agent, wherein the efficacy of the chemotherapeutic agent is enhanced relative to the efficacy of the chemotherapeutic agent alone.

In one preferred embodiment, the invention further provides a method of treating cancer, comprising the step of administering to a subject in need thereof an effective amount of an aldehyde-releasing compound prior to, together with, or subsequent to the administration of a therapeutically-effective amount of a chemotherapeutic agent, wherein the efficacy of the chemotherapeutic agent is enhanced relative to the efficacy of the chemotherapeutic agent alone.

In a second aspect, the invention provides a method of preferentially forming a chemotherapeutic agent-DNA adduct, comprising the step of administering to a subject in need thereof an effective amount of an aldehyde-releasing compound prior to, together with, or subsequent to the administration of a therapeutically-effective amount of a chemotherapeutic agent, wherein the chemotherapeutic agent more readily forms and/or increasingly forms, DNA adducts than compared to the chemotherapeutic agent alone.

Preferably the chemotherapeutic agent is an anthracycline such as Adriamycin, daunomycin, idarubicin or epirubicin, or an anthracenedione such as mitoxantrone. Adriamycin is particularly preferred.

The aldehyde releasing compound may be any compound that releases an aldehyde in situ. Aldehyde is released by decomposition of the compound or by hydrolysis of the compound by intracellular esterases. It is to be understood that the term aldehyde releasing compound should be interpreted broadly so as to include compounds that undergo a reaction in situ to form another compound that is then hydrolysed or decomposes to form an aldehyde. The aldehyde is usually released with at least one further compound, such as an acid.

Compounds that may release aldehyde in a given environment require at least one —CHR— unit with groups immediately adjacent to this unit that decompose or hydrolyse in situ. Accordingly, this term encompasses a very broad range of compounds, including:

(a) those compounds known to release aldehyde (particularly formaldehyde), such as hexamethylmelamine (altretamine) and hexamethylenetetramine (see for instance Ashby and Lefevre, 1982)

(b) diester compounds containing an ester to each side of the —CHR— unit, (ie R^{a}C(═O)O—CHR—OC(═O)R^{b}, wherein R^{a} and R^{b} independently have the same definitions as R¹ referred to below) including but not limited to compounds disclosed in U.S. Pat. No. 6,110,970, U.S. Pat. No. 6,040,342, U.S. Pat. No. 6,043,277, U.S. Pat. No. 5,710,176, U.S. Pat. No. 5,200,553, U.S. Pat. No. 6,130,248 and U.S. Pat. No. 6,043,389.

(c) compounds containing any two acid ester groups to either side of the —CHR— unit, such as compounds containing a carboxylic acid ester group and an ester based on an acid of phosphorous [eg (R′O)₂—P(═O), R′,R″O—P(═O) or R′₂—P(═O)] to either side of the —CHR— unit, including but not limited to compounds disclosed in U.S. Pat. No. 6,030,961

(d) compounds containing groups to either side of the —CHR— unit that will undergo hydrolysis (eg enzymatic hydrolysis) or decomposition to release formaldehyde, including compounds of the formula (I)

X—CHR—Y (I)

wherein:

X— and/or Y— is a group that can be converted to OH, NH or SH in situ, so that upon hydrolysis or decomposition the compound releases an aldehyde; and preferably X and Y are each independently —OR¹; —NHR²; —NR³R⁴; —SR⁵; —OAcyl; —SAcyl, a phosphorous acid radical, or a phosphoramide radical, or one of X and Y may be a halogen or hydrogen;

in which

R¹, R², and R⁵ are each independently optionally substituted alkyl, optionally substituted alkenyl, optionally substituted alkynyl, optionally substituted aryl, optionally substituted heteroaryl, optionally substituted aralkyl, optionally substituted aralkenyl or optionally substituted aralkynyl group, and

R³ and R⁴ each independently have the same definitions as R¹, R² and R⁵ above, or R³ and R⁴ may together with the nitrogen atom form an optionally substituted heterocyclic ring (eg a morpholine ring); and

(e) one of the new aldehyde-releasing compounds described in further detail below.

It is to be noted that the compounds of formula (I) above include within their scope the compounds set out in paragraphs a, b and c.

The term “alkyl” used either alone or in a compound word such as Optionally substituted alkyl or “optionally substituted cycloalkyl” denotes straight chain, branched or mono- or poly-cyclic alkyl, preferably C1-30 alkyl or cycloalkyl. Examples of straight chain and branched alkyl include methyl, ethyl, propyl, isopropyl, butyl, isobutyl, sec-butyl, tert-butyl, amyl, isoamyl, sec-amyl, 1,2-dimethylpropyl, 1,1-dimethylpropyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimetylpentyl, 1,2-dimethylpentyl, 1,3-dimethylpentyl, 1,4-dimethylpentyl, 1,2,3-trimethylbutyl, 1,1,2-trimethylbutyl, nonyl, 1-, 2-, 3-, 4-, 5-, 6- or 7-methyloctyl, 1-, 2-, 3-, 4- or 5-ethylheptyl, 1-2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-; 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. Examples of cyclic alkyl include cyclopropyl, cyclobutyl, cyclopentyl, cyclohexyl, cycloheptyl, cyclooctyl, cyclononyl and cyclodecyl and the like.

The term “alkenyl” used either alone or in compound words such as “alkenyloxy” denotes groups formed from straight chain, branched or cyclic alkenes including ethylenically mono-, di- or poly-unsaturated alkyl or cycloalkyl groups as defined above, preferably C2-20 alkenyl. Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4,pentadienyl, 1,3-cyclopentadienyl, 1,3-hexadienyl, 1,4-hexadienyl, 1,3-cyclohexadienyl, 1,4-cyclohexaidenyl, 1,3-cycloheptadienyl, 1,3,5-cycloheptatrienyl and 1,3,5,7-cyclooctatetraenyl.

The term “alkynyl” used either alone or in compound words such as “alkynyloxy” denotes groups formed from straight chain, branched or cyclic alkynes including ethylynically mono-, di- or poly-unsaturated alkyl or cycloalkyl groups as defined above, preferably C₂₋₂₀ alkynyl. The alkynyl preferably contains between 1 and 6 triple bonds. Examples of alkynyl include acetylenyl, prop-2-ynyl, pent-3-ynyl, hex-5-ynyl, 5-ethyldodec-3,6-diynyl, and the like.

The term “alkoxy” used either alone or in compound words such as “optionally substituted alkoxy” denotes straight chain or branched alkoxy, preferably C1-30 alkoxy. Examples of alkoxy include methoxy, ethoxy, n-propyloxy, isopropyloxy and the different butoxy isomers.

The term “acyl” used either alone or in compound words such as “optionally substituted acyl” or “optionally substituted acyloxy” denotes carbamoyl, aliphatic acyl group and acyl group containing an aromatic ring, which is referred to as aromatic acyl or a heterocyclic ring which is referred to as heterocyclic acyl, preferably C1-30 acyl. Examples of acyl include carbamoyl; straight chain or branched alkanoyl such as formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, pentadecanoyl, hexadecanoyl, heptadecanoyl, octadecanoyl, nonadecanoyl and icosanoyl; alkoxycarbonyl such as methoxycarbonyl, ethoxycarbonyl, t-butoxycarbonyl, t-pentyloxycarbonyl and heptyloxycarbonyl; cycloalkylcarbonyl such as cyclopropylcarbonyl cyclobutylcarbonyl, cyclopentylcarbonyl and cyclohexylcarbonyl; alkylsulfonyl such as methylsulfonyl and ethylsulfonyl; alkoxysulfonyl such as methoxysulfonyl and ethoxysulfonyl; aroyl such as benzoyl, toluoyl and naphthoyl; aralkanoyl such as phenylalkanoyl (e.g. phenylacetyl, phenylpropanoyl, phenylbutanoyl, phenylisobutyl, phenylpentanoyl and phenylhexanoyl) and naphthylalkanoyl (e.g. naphthylacetyl, naphthylpropanoyl and naphthylbutanoyl); aralkenoyl such as phenylalkenoyl (e.g. phenylpropenoyl, phenylbutenoyl, phenylmethacrylyl, phenylpentenoyl and phenylhexenoyl and naphthylalkenoyl (e.g. naphthylpropenoyl, naphthylbutenoyl and naphthylpentenoyl); aralkoxycarbonyl such as phenylalkoxycarbonyl (e.g. benzyloxycarbonyl); aryloxycarbonyl such as phenoxycarbonyl and naphthyloxycarbonyl; aryloxyalkanoyl such as phenoxyacetyl and phenoxypropionyl; arylcarbamoyl such as phenylcarbamoyl; arylthiocarbamoyl such as phenylthiocarbamoyl; arylglyoxyloyl such as phenylglyoxyloyl and naphthylglyoxyloyl; arylsulfonyl such as phenylsulfonyl and naphthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolylglyoxyloyl and thienylglyoxyloyl.

The term “aryl” used either alone or in compound words such as “optionally substituted aryl”, “optionally substituted aryloxy” or “optionally substituted heteroaryl” denotes single, polynuclear, conjugated and fused residues of aromatic hydrocarbons or aromatic heterocyclic ring systems. Examples of aryl include phenyl, biphenyl, terphenyl, quaterphenyl, phenoxyphenyl, naphtyl, tetrahydronaphthyl, anthracenyl, dihydroanthracenyl, benzanthracenyl, dibenzanthracenyl, phenanthrenyl, fluorenyl, pyrenyl, indenyl, azulenyl, chrysenyl, pyridyl, 4-phenylpyridyl, 3-phenylpyridyl, thienyl, furyl, pyrryl, pyrrolyl, furanyl, imadazolyl, pyrrolydinyl, pyridinyl, piperidinyl, indolyl, pyridazinyl, pyrazolyl, pyrazinyl, thiazolyl, pyrimidinyl, quinolinyl, isoquinolinyl, benzofuranyl, benzothienyl, purinyl, quinazolinyl, phenazinyl, acridinyl, benzoxazolyl, benzothiazolyl and the like. Preferably, the aromatic heterocyclic ring system contains 1 to 4 heteratoms independently selected from N, O and S and containing up to 9 carbon atoms in the ring.

The term “heterocyclyl”used either alone or in compound words such as “optionally substituted saturated or unsaturated heterocyclyl” denotes monocyclic or polycyclic heterocyclyl groups containing at least one heteroatom atom selected from nitrogen, sulphur and oxygen. Suitable heterocyclyl groups include N-containing heterocyclic groups, such as, unsaturated 3 to 6 membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl or tetrazolyl;

saturated 3 to 6-membered heteromonocyclic groups containing 1 to 4 nitrogen atoms, such as, pyrrolidinyl, imidazolidinyl, piperidino or piperazinyl;

unsaturated condensed heterocyclic groups containing 1 to 5 nitrogen atoms, such as indolyl, isoindolyl, indolizinyl, benzimidazolyl, quinolyl, isoquinolyl, indazolyl, benzotriazolyl or tetrazolopyridazinyl;

unsaturated 3 to 6-membered heteromonocyclic group containing an oxygen atom, such as, pyranyl or furyl;

unsaturated 3 to 6-membered heteromonocyclic group containing 1 to 2 sulphur atoms, such as, thienyl;

unsaturated 3 to 6-membered heteromonocyclic group containing 1 to 2 oxygen atoms and 1 to 3 nitrogen atoms, such as, oxazolyl, isoxazolyl or oxadiazolyl;

saturated 3 to 6-membered heteromonocyclic group containing 1 to 2 oxygen atoms and 1 to 3 nitrogen atoms, such as, morpholinyl;

unsaturated condensed heterocyclic group containing 1 to 2 oxygen atoms and 1 to 3 nitrogen atoms, such as, benzoxazolyl or benzoxadiazolyl;

unsaturated 3 to 6-membered heteromonocyclic group containing 1 to 2 sulphur atoms and 1 to 3 nitrogen atoms, such as, thiazolyl or thiadiazolyl;

saturated 3 to 6-membered heteromonocyclic group containing 1 to 2 sulphur atoms and 1 to 3 nitrogen atoms, such as, thiazolidinyl; and

unsaturated condensed heterocyclic group containing 1 to 2 sulphur atoms and 1 to 3 nitrogen atoms, such as benzothiazolyl or benzothiadiazolyl.

The terms “aralkyl”, “aralkenyl” and “aralkynyl” refer to alkyl, alkenyl and alkynyl, respectively, substituted with an aryl or heteroaryl group.

In this specification “optionally substituted” means that a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy, mercapto, alkylthio, benzylthio, acylthio, phosphorus-containing groups and the like. Preferred substituents in each instance are alkoxy, alkylthio, hydroxy, halogen, cyano, azido, alkylsulfonyl, oxo and acyloxy, unless otherwise indicated.

“Halide” or “halo” denotes fluorine, chlorine, bromine or iodine, and preferably F or Cl.

The term “phosphorous acid radical” denotes one of the phosphorous acids such as (R⁶O)₂—P(═O), R⁷,R⁸⁰—P(═O) or R⁹₂—P(═O), in which R⁶, R⁷, R⁸ and R⁹ each independently have the same definitions as R¹ above.

The term “phosphoramide radical” is used in this specification in its broadest sense to refer to a radical containing a phosphorous-oxygen double bond, with the same phosphorous atom being bound to a nitrogen atom by a single bond. This nitrogen atom is the one through which the phosphoramide radical is connected to the —CHR— unit. Usually, the phosphoramide radical contains three nitrogen atoms bound to the phosphorous atom in addition to the double-bonded oxygen atom. An example is heptamethyl phosphoramido.

The aldehyde releasing compounds release one or more further compounds in addition to the aldehyde. The diester compounds referred to under paragraph (b) above release two acids, which, depending on the ester units, may be butyric acid, retinoic acid or any other acid. The compounds referred to under paragraph (c) above release a phosphorous acid.

In order to avoid any doubt whatsoever, the following explanation is provided of the mechanism by which aldehyde is released from suitable aldehyde releasing compounds in situ. Further information may also be found in Ashby and Lefevre (1982), the entire disclosure of which is incorporated herein by reference. This explanation will assist the person skilled in the art to comprehend the meaning of the term hydrolysable or decomposable group, and therefore the full range of compounds that will release formaldehyde in the given conditions.

The atom to either side of the —CHR— unit in the aldehyde-releasing compound may be any heteroatom such as O, N or S, or it can be a halogen, if the halogen can be replaced with a heteroatom by reaction in situ. There is a requirement that at least one of the groups to either side of the —CHR— unit must be converted to OH, NH or SH in situ. For instance, ClCH₂Cl where X═Y═Cl is a stable compound that would not decompose readily to release formaldehyde. On the other hand, the compound C₁-CH₂—OH (in which the heteroatom is O, and is connected to a hydrogen atom) is unstable and undergoes instantaneous decomposition to formaldehyde H₂C═O and HCl. In another example, MeO—CH₂—OMe is very stable, however in the presence of an acid one of the ether oxygen atoms is protonated, and the protonated compound is destabilised releasing formaldehyde and 2 MeOH moieties. This is also the pathway of decomposition of hexamethylenetetramine, which requires acidic conditions to protonate one nitrogen atom giving a —⁺NH—CH₂—N— intermediate that again hydrolyzes to give 6 formaldehyde and 4 NH₃ moieties.

It is noted that the heteroatom immediately next to the —CHR— cannot be part of an electron withdrawing group, as this stabilises the compound. Accordingly, MeCONH—CH₂—OH or MeCOCH—CH₂—NH, are too stable to be efficient sources of formaldehyde.

Preferably, R in the unit —CHR— is H or C1-4 alkyl, alkenyl or alkynyl. It is most preferred that aldehyde released be formaldehyde (ie the —CHR— unit in the aldehyde releasing compound is —CH₂—). Nevertheless, smaller aldehydes such as acetaldehyde, propanal, butanal and butenal (eg 2-butenal) may also be suited for use in combination therapies with certain chemotherapeutic agents, and therefore compounds that release these smaller aldehydes are to be considered to be within the broad concept of the invention.

The present Applicant has developed a new range of aldehyde releasing compounds that have been found to give surprisingly excellent results in adduct formation tests. One class of new aldehyde releasing compounds of formula (II) release more than one equivalent of aldehyde:

Z-(L-M′-CHR-M²)ₓ (II)

wherein:

x is an integer of 2 or more;

Z is a direct bond or a linking group of valency x;

L is either a direct bond or a spacer group;

R is H or C1-4 alkyl, alkenyl or alkynyl;

M¹ is a decomposable or hydrolysable group; and

M² is a second decomposable or hydrolysable group.

Z may be a direct bond (when x=2) or any group that can link the bracketed portions of the compound together (ie a Blinking groups). For example, Z may be N (x=3, or 2 if the nitrogen atom includes another substituent), P(═O), PO, O, S, an optionally substituted C1-20 alkylene, alkenylene or alkynylene chain, which may optionally be interspersed with one or more aryl or heteroaryl groups (which may also be optionally substituted) and/or one or more O, S or N atoms; or Z may be an optionally substituted heterogenous cyclic group, an aryl group or heteroaryl group.

The terms “alkylene”, “alkenylene” and “alkynylene” are the divalent radical equivalents of the terms “alkyl” “alkenyl” and “alkynyl”, respectively. The two bonds connecting the alkylene, alkenylene or alkynylene to the adjacent groups may come from the same carbon atom or different carbon atoms in the divalent radical.

Preferred optional substituents in the linker group Z are selected from halogen, oxy, hydroxy, alkoxy, alkylthio, cyano, azido, acyloxy, alkylsulphonyl, aryl and heteroaryl.

It will be understood that in addition to being a linking group, Z could have a second function. For example, Z could be a radical based on the chemotherapeutic agent itself.

The term “spacer group” is to be interpreted broadly so as to include any divalent organic group that separates the next adjacent groups from one another (eg groups Z and M¹). As a consequence, L may be any one of the groups outlined above for Z where Z has a valency of 2.

In the situation where x is 2, Z and L may each be a direct bond, such that M¹ of one of the bracketed groups (hereinafter referred to as “chain a”, and therefore M^{1a} refers to M¹ in chain a) is directly connected to M¹ of the second of the bracketed groups (referred to as “chain b”). For example, in one preferred embodiment of the invention, M^{1a}-M^{1b} is —O—C(═O)—C(═O)—C—O—. It is also to be noted that the hydrolysable groups M^{1a} and M^{1b} in this embodiment may form part of the one group. That is, -M^{1a}-M^{1b} could for example be —O—C(═O)—O—.

Each M² in the compound (ie M^{2a}, M^{2b}, etc) is independently any hydrolysable or decomposable group as described above in relation to the mechanism for the formation of aldehyde in situ, and in one preferred embodiment each M² has the same definition as X (or Y) outlined above.

Each M¹ in the compound (ie M^{1a}, M^{2a}, etc) is independently any hydrolysable or decomposable group as described above. M¹ is the divalent radical equivalent of M².

Each group L, M¹, R and M² in each chain (chains a, b etc) may be the same or different. Accordingly, the compounds may be symmetrical or unsymmetrical.

As a consequence of the above, the new compounds include compounds of the formula (III):

X′—CH₂—OOC-Z′-COO—CH₂—Y′ (III),

in which X′ and Y′ have the same definitions as X and Y described above for the compounds of formula (I), respectively, and Z′ has the same definition as Z described above for the compound of formula (II), where Z has a valency of 2.

In one embodiment of the compound of formula III, Z′ is an optionally substituted cyclic alkylidene, an optionally substituted cyclic alkenylidene, an optionally substituted cyclic alkynylidene, an optionally substituted heterocyclic group, an optionally substituted aryl or an optionally substituted heteroaryl group. Thus —OOC-Z′-COO— may be a fragment from a dicarboxy-substituted saturated or unsaturated cyclic diacid, which may be an alicyclic, heterocyclic, aromatic or heteroaromatic ring system, such as 1,3-dicarboxy-cyclohexane; phthalic acid; 2,5-dicarboxy-thiazole; 2,5-dicarboxy-tetrahydrofuran; 3,4-dicarboxy-thiophen; 3,4-dicarboxy-oxazolidine-2-one.

Where Z′ is a direct bond, —OOC-Z′-COO— may be a fragment derived from oxalic acid.

Where Z′ is alkylidene, —OOC-Z′-COO— may be a fragment derived for example from malonic acid, succinic acid or glutaric acid. Where Z′ is alkenylidene, —OOC-Z′-COO— may be a fragment derived for example from maleic acid or fumaric acid.

The present invention also provides a method of synthesising the new compound of formula (II) described above, the method including the step of reacting a compound from which the fragment Z-(L-M¹-), is derived, with a compound from which the fragment —CHR-M² is derived, to form the compound of formula (II). In the situation where the compound from which the fragment Z-(L-M¹-)ₓ is derived is an acid, the compound from which the fragment —CHR-M² is derived may be Hal-CHR-M², in which Hal refers to a halogen or another suitable leaving group. These two fragments can then be reacted together in the presence of a base.

The leaving group may be one of those disclosed in March, 1992, the entire disclosure of which is incorporated herein by reference.

Therapeutic effectiveness may be further improved by localisation of the aldehyde-releasing compounds to tumour tissues and/or sub-cellular compartments of tumour cells. Thus preferably the aldehyde-releasing compound is preferentially targeted to the tumour. This may be achieved by any suitable method, including but not limited to:

(a) coupling the aldehyde-releasing compound to a cellular or subcellular targeting sequence, such as a nuclear targeting sequence; and

(b) coupling the aldehyde-releasing compound to a tumour-localising component, such as an antibody or an antibody-derived fragment specific for a tumour cell marker.

As a consequence, the present invention also provides a compound which includes an aldehyde-releasing compound as described above coupled to a cellular or subcellular targeting sequence or a tumour-localising component.

Polyclonal or monoclonal antibodies may be used, and may be made by methods known in the art; preferably the antibody is a monoclonal antibody. Suitable antibody-derived fragments include ScFv fragments; suitable tumour cell markers are tumour-specific cell surface or intracellular antigens.

The aldehyde-releasing compound preferably releases aldehyde mainly in tumour tissues. The intracellular level of aldehyde can be further enhanced by reducing the level of aldehyde detoxifying agents in the tissues. The aldehyde detoxifying agents present in the tissues include GSH, GSH-dependent formaldehyde dehydrogenase, mitochondrial aldehyde dehydrogenase (non-glutathione-dependent) and other alcohol dehydrogenases. These agents detoxify the formaldehyde by oxidising the formaldehyde. Inhibitors of these enzymes include buthionine sulfoximine (BSO) (which lowers glutathione (GSH) levels by inhibiting gamma-glutamyl synthetase), Daidzin and crotonaldehyde (which inhibit mitochondrial aldehyde dehydrogenase—see Keung and Vallee, 1993 and Dicker and Cederbaum, 1984, respectively), semicarbazides, dimedone and resveratrol (which all act by direct binding to formaldehyde), diethyl maleate, phorone and cyanamide.

It follows from the above that the method of the present invention may involve administering a compound that reduces the intracellular level of one or more aldehyde detoxifying agents. This compound may be separate to the aldehyde-releasing compound, or otherwise a single compound may be both the aldehyde-releasing compound and the compound that reduces the intracellular level of the aldehyde detoxify

In one particular embodiment of the present invention, there is provided an aldehyde releasing compound including a radical based on an inhibitor of an aldehyde detoxifying agent, which aldehyde releasing compound releases said inhibitor and an aldehyde on hydrolysis or decomposition in situ. It is to be understood that the agent and the aldehyde may be one and the same in this embodiment of the invention, as is explained by way of example below.

Preferably the inhibitor of an aldehyde detoxifying agent is selected from the group consisting of inhibitors of gamma-glutamyl synthetase and inhibitors of alcohol and aldehyde dehydrogenases. Preferably the inhibitor is buthionine sulfoximine or crotonaldehyde, or a derivative of one of these inhibitors.

This class of new compounds therefore includes compounds of the formula (IV)

M³-CHR-M⁴ (IV)

where M³ and M⁴ are each independently a hydrolysable or decomposable group, and

M³ and/or M⁴ and/or R is a radical based on an inhibitor of an aldehyde detoxifying agent.

The term “based on” in this context means that the radical is selected such that when the aldehyde-releasing compound is decomposed or hydrolysed, the portion of the compound that contains the specified radical is decomposed or hydrolysed to form said inhibitor. In one embodiment of this aspect of the invention, M³ is a BSO radical. Accordingly, aldehyde-releasing compounds of this class include the oxymethylesters of BSO which release formaldehyde and BSO on cellular hydrolysis, the BSO functioning to limit formaldehyde detoxification. In another embodiment, R is a radical based on crotonaldehyde (ie a 2-butenyl radical) such that crotonaldehyde is released on hydrolysis. In a preferred embodiment, M⁴ has the same definition as Y in the compound of formula (I) above.

The present invention also provides a method of synthesising an aldehyde releasing compound of the formula M³-CHR-M⁴ (as defined above) in which M³ is a radical based on an inhibitor of an aldehyde detoxifying agent, the method comprising the step of coupling the radical based on said inhibitor to a radical —CHR-M⁴. In the situation where the inhibitor is a carboxylic acid (eg when the inhibitor is BSO), the method may involve the step of reacting the inhibitor with the compound Hal-CHR-M⁴, wherein R and M are as defined above, and Hal is a halogen or halogen-like group (eg a leaving group such as a tosylate group) in the presence of a base.

The new classes of compounds of formulae (II) and (III) referred to above should be understood to include within their scope compounds that release one or more equivalents of an inhibitor of an aldehyde detoxifying agent, together with two or more equivalents of aldehyde. In this embodiment, M² should be understood to include BSO. In addition, in the case where the agent that enhances intracellular levels of aldehyde is crotonaldehyde, one or more of the aldehydes released in the molecule could be crotonaldehyde (ie R (eg R^{a}) is —CH═CHCH₃).

In a fourth aspect, the invention provides a composition comprising

(a) a chemotherapeutic agent which is a primary or secondary amine, and

(b) an aldehyde-releasing compound which upon hydrolysis releases one or more equivalents of formaldehyde, together with a pharmaceutically-acceptable carrier.

The aldehyde-releasing compound may be any one of the known aldehyde-releasing agents, or one of the new aldehyde-releasing agents described above.

In a fifth aspect, the invention provides a composition comprising one or more of the new aldehyde-releasing compounds as defined above, together with a pharmaceutically-acceptable carrier.

In a sixth aspect the invention provides for the use of an aldehyde-releasing compound in the manufacture of a medicament for the treatment and/or prophylaxis of cancer. Preferably the aldehyde-releasing compound is one of the new aldehyde-releasing compounds described above.

In a seventh aspect the invention provides a method of treating cancer comprising the steps of:

(a) determining the optimum time of administration of a therapeutically effective amount of an aldehyde-releasing compound relative to the administration of a chemotherapeutic agent wherein the optimum time is determined by the number of DNA adducts formed;

(b) determining the amount of aldehyde-releasing compound relative to the amount of chemotherapeutic agent;

(c) from step (a) and (b) determining the amount and timing of delivery of aldehyde-releasing compound and chemotherapeutic agent and administering to a patient in need thereof.

The major potential benefits of the method of the invention are:

(1) The higher level of tumour cell killing resulting from the combined use of an active chemotherapeutic agent (eg anthracycline or anthracenedione) and an aldehyde-releasing compound enables the chemotherapeutic agent to be used at lower doses in order to achieve the same level of cell killing, hence decreasing the level of undesired side effects of the chemotherapeutic agent (eg cardiotoxicity).

(2) The co-administration of an anthracycline or anthracenedione with an aldehyde-releasing compound is predicted to be effective against anthracycline-resistant cells, because the chemotherapeutic agent will form adducts with DNA rather than be subjected to active efflux, or other detoxifying mechanisms.

(3) Targeting of the aldehyde-releasing compound to the tumour localises the formation of chemotherapeutic agent-DNA adducts preferentially to tumours, and hence localises the cell killing primarily to the tumour, thus minimising the side effects which result from damage to normal tissues.

(4) The potentiation of adduct formation can be utilized as a diagnostic tool for the optimisation of chemotherapeutic agent dosage and the prediction of tumour response to the treatment with the aldehyde-releasing compound/chemotherapeutic agent combinations.

### BRIEF DESCRIPTION OF THE FIGURES

FIG. 1 illustrates the formation of DNA adducts in IMR-32 (human neuroblastoma) and MCF-7 (human breast adenocarcinoma) cells in the presence of AN-9 and Adriamycin. IMR-32 human neuroblastoma cells (A and B) and MCF-7 human breast adenocarcinoma cells (C and D) were treated with Adriamycin for 2 hr, followed by a further 2 hr incubation with a 25-fold excess of AN-9 (▪), or with AN-9 for 2 hr followed by a further 4 hr incubation with Adriamycin (□).

FIG. 2 shows the time-dependent formation of adducts in the mitochondrial genome (A) and DHFR gene (B).

FIG. 3 shows the effect on enhanced adduct formation of the order of addition of Adriamycin and AN-9. Adduct/crosslinking levels for both mitochondrial (A) and nuclear (B) DNA are shown.

FIG. 4 shows that reversing the order of addition results in diminished adduct formation. Adduct/crosslinking levels for both mitochondrial (A) and nuclear (B) DNA are shown.

FIG. 5 shows the effect of AN-9 on barminomycin-induced crosslinking of mitochondrial (A) and nuclear DNA (B). IMR-32 cells were treated with barminomycin alone (0-20 DM, ▪) for 2 hr, or barminomycin for 0.5 hr followed by a further 1.5 hr incubation with AN-9 using a 12,500-fold excess of AN-9 at each barminomycin concentration ( ), or AN-9 for 2 hr followed by a further 2 hr incubation with barminomycin (□).

FIG. 6 illustrates the schedule-dependent potentiation of adduct formation by AN-9.

FIG. 7 shows the effect of adding AN-9 many hours before Adriamycin, and also shows the effect of butyric acid.

FIG. 8 compares adduct formation by AN-9 and by aldehyde-releasing compounds which do not release formaldehyde.

FIG. 9 shows the concentration dependence of the effect of AN-9.

FIG. 10 shows the ability of hexamethylenetetramine to facilitate Adriamycin adducts.

FIG. 11 shows the stability of AN-9 induced Adriamycin adducts in cells.

FIG. 12 shows the sequence specificity of AN-9 induced Adriamycin-DNA adducts in cells.

FIG. 13 shows the binding of AN-9 induced Adriamycin adducts to RNA, DNA and protein.

### DETAILED DESCRIPTION OF THE INVENTION

Before the present compounds, compositions, and methods are described, it is understood that this invention is not limited to the particular materials and methods described, as these may vary. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “an,” and “the” include plural reference unless the context clearly dictates otherwise. Thus, for example, a reference to “a compound” includes compounds of similar formula and equivalents thereof known to those skilled in the art, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The methods and compounds of the invention are useful for enhancing the efficacy of chemotherapeutic agents such as, for example, anti-cancer agents like Adriamycin, daunomycin, idarubicin or epirubicin, or an anthracenedione such as mitoxantrone. Increased efficacy may be measured as an increase in bioavailability, increase in antiproliferative activity or decreased toxic side effect of the chemotherapeutic agent. By increasing bioavailability or antiproliferative activity or reducing toxic side effects associated with the use of chemotherapeutic agents, the invention satisfies some of the shortcomings of current therapeutic modalities.

The description that follows makes use of a number of terms used in pharmaceutical chemistry and cell biology. In order to provide a clear and consistent understanding of the specification and claims, including the scope given such terms, the following definitions are provided.

The term “endogenous” means originating within the subject, cell, or system being studied. Accordingly, supplementing the endogenous levels of aldehyde means that a compound or compounds is/are administered to a subject such that the total amount of aldehyde in the subject is higher than normally present. Increasing the endogenous levels of aldehyde means that a compound or compounds is/are administered to a subject where the compound or compounds increase the production of aldehyde by the subjects cells or tissue, thereby effectively increasing the total amount of aldehyde in the subject. The endogenous levels of aldehyde may also be effectively increased by decreasing the detoxification of aldehyde. For example, a compound or compounds of the invention when administered to a subject may decrease the rate of detoxification of endogenous aldehyde by inhibiting the effect of detoxifying agents.

The term “hydrocarbon” refers to alkyl, alkenyl or alkynyl groups as defined above in relation to the compounds of formula (I).

It will be appreciated by those skilled in the art that the compounds of the invention may be modified to provide pharmaceutically acceptable derivatives thereof at any of the functional groups in the compounds of formula (I).

The term “pharmaceutically acceptable derivative” means any pharmaceutically acceptable salt, ester or salt of such ester of a compound of formula (I) or any other compound which, upon administration to the recipient, is capable of providing a compound of formula (I) or a biologically active metabolite or residue thereof. Of particular interest as derivatives are compounds modified at the sialic acid carboxy or glycerol hydroxy groups, or at the amino and guanidino groups.

Pharmaceutically acceptable salts of the compounds of formula (I) include those derived from pharmaceutically acceptable inorganic and organic acids and bases. Examples of suitable acids include hydrochloric, hydrobromic, sulphuric, nitric, perchloric, fumaric, maleic, phosphoric, glycolic, lactic, salicylic, succinic, toluene-p-sulphonic, tartaric, acetic, citric, methanesulphonic, formic, benzoic, malonic, naphthalene-2-sulphonic and benzenesulphonic acids. Other acids such as oxalic acid, while not in themselves pharmaceutically acceptable, may be useful in the preparation of salts useful as intermediates in obtaining compounds of the invention and their pharmaceutically acceptable acid addition salts.

Salts derived from appropriate bases include alkali metal (eg. sodium), alkaline earth metal (eg. magnesium), ammonium, and NR₄⁺ (where R is C₁₋₄ alkyl) salts.

The term “toxic side effects” or “side effects” means the deleterious, unwanted effects of chemotherapy on the subject's normal, non-diseased tissues and organs. For example, toxic side effects may include bone marrow suppression (including neutropenia), cardiac toxicity, hair loss, gastrointestinal toxicity (including nausea and vomiting), neurotoxicity, lung toxicity and asthma.

The term “subject” as used herein refers to any animal having a disease or condition which requires treatment with a chemotherapeutic agent. The chemotherapeutic agent may also have bioavailability problems or causes toxic side effects. Preferably the subject is suffering from a cellular proliferative disorder (eg., a neoplastic disorder). Subjects for the purposes of the invention include, but are not limited to, mammals (eg., bovine, canine, equine, feline, porcine) and preferably humans.

By “cell proliferative disorder” is meant that a cell or cells demonstrate abnormal growth, typically aberrant growth, leading to a neoplasm, tumour or a cancer.

Cell proliferative disorders include, for example, cancers of the breast, lung, prostate, kidney, skin, neural, ovary, uterus, liver, pancreas, epithelial, gastric, intestinal, exocrine, endocrine, lymphatic, haematopoietic system or head and neck tissue.

Generally, neoplastic diseases are conditions in which abnormal proliferation of cells results in a mass of tissue called a neoplasm or tumour. Neoplasms have varying degrees of abnormalities in structure and behaviour. Some neoplasms are benign while others are malignant or cancerous. An effective treatment of neoplastic disease would be considered a valuable contribution to the search for cancer preventive or curative procedures.

The methods of this invention involve in one embodiment, (1) the administration of an aldehyde-releasing compound, prior to, together with, or subsequent to the administration of a chemotherapeutic agent; or (2) the administration of a combination of aldehyde-releasing compounds and a chemotherapeutic agent.

As used herein, the term “effective amount” is meant an amount of an aldehyde-releasing compound of the present invention effective to increase the efficacy of a chemotherapeutic agent in order to yield a desired therapeutic response. For example, to increase the efficacy of a chemotherapeutic agent by increasing bioavailability or by preventing or reducing the toxic side effects caused by the use of chemotherapeutic agents.

The term therapeutically-effective amount” means an amount of a chemotherapeutic agent to yield a desired therapeutic response. For example, treat or prevent a neoplastic disease.

The specific “therapeutically-effective amount” will, obviously, vary with such factors as the particular condition being treated, the physical condition of the subject, the type of animal being treated, the duration of the treatment, the nature of concurrent therapy (if any), and the specific formulations employed and the structure of the chemotherapeutic agent or its derivatives.

As used herein, a “pharmaceutical carrier” is a pharmaceutically-acceptable solvent, suspending agent or vehicle for delivering the aldehyde-releasing compound and/or chemotherapeutic agent to the animal or human. The carrier may be liquid or solid and is selected with the planned manner of administration in mind.

As used herein, “cancer” refers to all types of cancers or neoplasm or malignant tumours found in marnmals. Cancer includes sarcomas, lymphomas and other cancers. The following types are examples, but are not intended to be limited to these particular types of cancers: prostate, colon, rectal, breast, both the MX-1 and the MCF lines, pancreatic, neuroblastoma, rhabdomysarcoma, bone, lung, murine, melanoma, leukemia, pancreatic, melanoma, ovarian, brain, head & neck, kidney, mesothelioma, sarcoma, Kaposi's sarcoma, stomach, uterine and lymphoma.

As used herein the term “cell” includes but is not limited to mammalian cells (eg., mouse cells, rat cells or human cells).

The aldehyde-releasing compound and/or chemotherapeutic agents may be administered orally, topically, or parenterally in dosage unit formulations containing conventional non-toxic pharmaceutically acceptable carriers, adjuvants, and vehicles. The term parenteral as used herein includes subcutaneous injections, aerosol, intravenous, intramuscular, intrathecal, intracranial, injection or infusion techniques.

The present invention also provides suitable topical, oral, and parenteral pharmaceutical formulations for use in the novel methods of treatment of the present invention. The compounds of the present invention may be administered orally as tablets, aqueous or oily suspensions, lozenges, troches, powders, granules, emulsions, capsules, syrups or elixirs. The composition for oral use may contain one or more agents selected from the group of sweetening agents, flavouring agents, colouring agents and preserving agents in order to produce pharmaceutically elegant and palatable preparations. The tablets contain the active ingredient in admixture with non-toxic pharmaceutically acceptable excipients which are suitable for the manufacture of tablets.

These excipients may be, for example, (1) inert diluents, such as calcium carbonate, lactose, calcium phosphate or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed. Coating may also be performed using techniques described in the U.S. Pat. Nos. 4,256,108; 4,160,452; and 4,265,874 to form osmotic therapeutic tablets for control release.

The aldehyde-releasing compounds as well as the chemotherapeutic agents useful in the methods of the invention can be administered, for in vivo application, parenterally by injection or by gradual perfusion over time independently or together. Administration may be intravenously, intra-arterial, intraperitoneally, intramuscularly, subcutaneously, intracavity, or transdermally. For in vitro studies the agents may be added or dissolved in an appropriate biologically acceptable buffer and added to a cell or tissue.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, growth factors and inert gases and the like.

It is envisioned that the invention can be used to increase the efficacy of chemotherapeutic agents used to treat cell proliferative disorders, including, for example, neoplasms, cancers (eg., cancers of the breast, lung, prostate, kidney, skin, neural, ovary, uterus, liver, pancreas, epithelial, gastric, intestinal, exocrine, endocrine, lymphatic, haematopoietic system or head and neck tissue), fibrotic disorders and the like.

The methods and compounds of the invention may also be used to increase the efficacy of chemotherapeutic agents used to treat other diseases such as neurodegenerative disorders, hormonal imbalance and the like.

Generally, the terms “treating”, “treatment” and the like are used herein to mean affecting a subject, tissue or cell to obtain a desired pharmacologic and/or physiologic effect. The effect may be prophylactic in terms of completely or partially preventing a disease or sign or symptom thereof, and/or may be therapeutic in terms of a partial or complete cure of a disease. “Treating” as used herein covers any treatment of, or prevention of a disease in a vertebrate, a mammal, particularly a human, and includes: (a) preventing the disease from occurring in a subject that may be predisposed to the disease, but has not yet been diagnosed as having it; (b) inhibiting the disease, ie., arresting its development; or (c) relieving or ameliorating the effects, ie., cause regression of the effects of the disease.

The invention includes various pharmaceutical compositions useful for treating a disease. The pharmaceutical compositions according to one embodiment of the invention are prepared by bringing an aldehyde-releasing compound, analogue, derivative or salt thereof and one or more chemotherapeutic agents into a form suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's, 1975, and The National Formulary, 1975, the contents of which are hereby incorporated by reference. The pH and exact concentration of the various components of the pharmaceutical composition are adjusted according to routine skills in the art—see Goodman and Gilman.

The pharmaceutical compositions are preferably prepared and administered in dose units. Solid dose units are tablets, capsules and suppositories. For treatment of a subject, depending on activity of the chemotherapeutic agent, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of particular disease. Various considerations are described, eg. in Langer, 1990. Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain the active materials in admixture with excipients suitable for the manufacture of aqueous suspension. Such excipients may be (1) suspending agent such as sodium carboxymethyl cellulose, methyl cellulose, hydroxypropylmethylcellulose, sodium alginate, polyvinylpyrrolidone, gum tragacanth and gum acacia; (2) dispersing or wetting agents which may be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleagenous suspension. This suspension may be formulated according to known methods using those suitable dispersing or wetting agents and suspending agents which have been mentioned above. The sterile injectable preparation may also a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono-or diglycerides. In addition, fatty acids such as oleic acid find use in the preparation of injectables.

Aldehyde-releasing compounds may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Dosage levels of the aldehyde-releasing compounds of the present invention are of the order of about 0.5 mg to about 20 mg per kilogram body weight, with a preferred dosage range between about 5 mg to about 20 mg per kilogram body weight per day (from about 0.3 g to about 3 g per patient per day). The amount of active ingredient that may be combined with the carrier materials to produce a single dosage will vary depending upon the host treated and the particular mode of administration. For example, a formulation intended for oral administration to humans may contain about 5 mg to 1 g of an active compound with an appropriate and convenient amount of carrier material which may vary from about 5 to 95 percent of the total composition. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, chemotherapeutic agent combination and the severity of the particular disease undergoing therapy.

In addition, some of the compounds of the instant invention may form solvates with water or common organic solvents. Such solvates are encompassed within the scope of the invention.

The compounds of the present invention may additionally be combined with other compounds to provide an operative combination. It is intended to include any chemically compatible combination of chemotherapeutic agents or aldehyde-releasing compound, as long as the combination does not eliminate the ability of the aldehyde-releasing compound of this invention to increase efficacy of the chemotherapeutic agents.

The invention will now be further described by way of reference only to the following non-limiting examples. It should be understood, however, that the examples following are illustrative only, and should not be taken in any way as a restriction on the generality of the invention described above. In particular, while the invention is described in detail in relation to cancer, it will be clearly understood that the findings herein are not limited to treatment of cancer. For example, aldehyde-releasing compounds may be used in combination with chemotherapeutic agents used in the treatment of other diseases.

### EXAMPLE 1

### Synergism Between Adriamycin and AN-9 in IMR-32 and PC-3 Cells

Previous data had suggested that there was a synergistic interaction between various anthracyclines and AN-9 in producing a cytotoxic response in mouse Mm-A cells (Kasukabe et al., 1997). In order to test the synergistic relationship between Adriamycin and AN-9 in human cells, cytotoxicity studies were performed in human neuroblastoma and prostate adenocarcinoma cells in culture.

IMR-32 human neuroblastoma and PC-3 prostate adenocarcinoma cells (100 μL at a density of 5×10⁴ cells/mL) were seeded in tissue culture 96 well plates (in triplicate) for 48 hr. They were exposed to different concentrations of the drugs at the specified ratio and times. Viability was determined by neutral-red assay as described by Kopf-Maier and Kolon (1992). The mean value obtained from 3 wells was calculated, and IC₅₀ values were derived from non-linear regression of the adjusted Y (% control viability) values against the log concentration of the compounds. Combination Index (CI) values were evaluated according to the classical isobologram equation:

CI=(D)(Dx), +(D)₂/(Dx)₂

where Dx is the dose of one compound alone required to produce an effect, and (D)₁ and (D)₂ are the doses of both compounds that produce the same effect. From this analysis the combined effects of two compounds can be assessed as either additive (or zero) interaction indicated by CI=1, synergistic interaction as indicated by CI<1, or antagonistic interaction as indicated by CI>1. The results are shown in Table 1.

TABLE 1

CI

| Order of addition | AN-9:Adr | IMR-32 | PC-3 |
|---|---|---|---|
| simultaneous | 25:1 | 0.25 | 0.88 |
| simultaneous | 50:1 | 0.41 | 0.61 |
| (AN-9)-1 | 25:1 | 2.5 | 2.0 |
| (AN-9)-1 | 50:1 | 2.0 | 1.66 |
| Adr-1 | 25:1 | 0.56 | 0.62 |
| Adr-1 | 50:1 | 0.88 | 0.49 |

Compounds were added to cells simultaneously at ratios of AN-9:Adr of 25:1 and 50:1. The combination exhibited synergy using both ratios. In contrast, when AN-9 was added 16 hr prior to Adriamycin (AN-9-1), antagonism was observed. However, good synergy was maintained when Adriamycin was added 16 hr prior to AN-9 (Adr-1). These results were significant, since they implied a mechanism of cytotoxicity which was highly dependent on a defined sequence of interactions between the two drugs.

### EXAMPLE 2

### Formation of DNA Adducts in IMR-32 (Human Neuroblastoma) and MCF-7 (Human Breast Adenocarcinoma) Cells in the Presence of AN-9 and Adriamycin

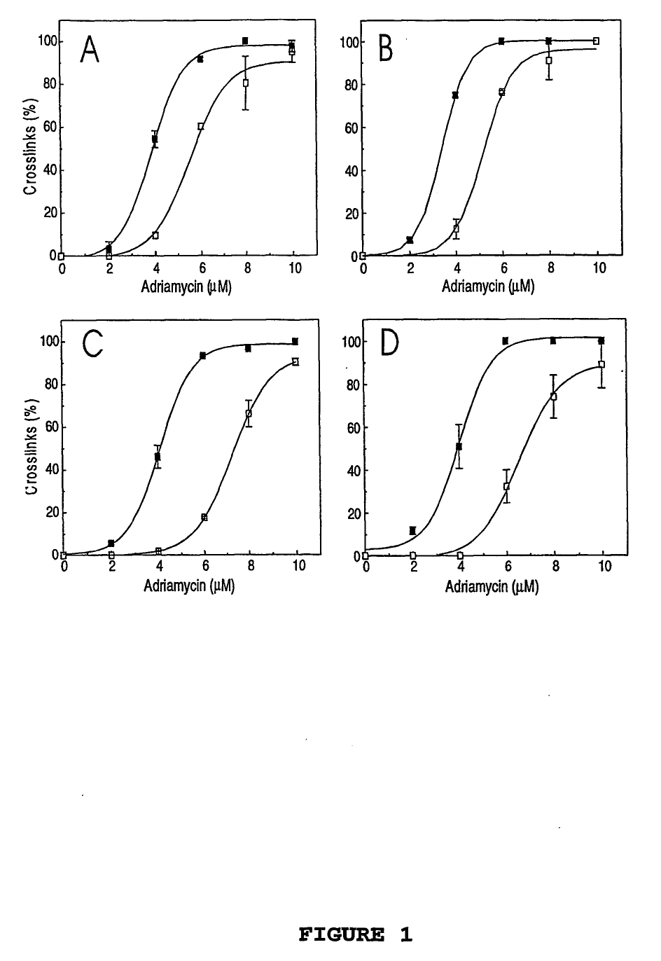

We sought to investigate the level of DNA adducts formed under various treatment conditions, since we speculated that the peculiar effectiveness of the Adriamycin/AN-9 combination might be due to increased levels of DNA damage in the form of DNA adducts. IMR-32 human neuroblastoma cells (A and B) and MCF-7 human breast adenocarcinoma cells (C and D) were treated with Adriamycin for 2 hr (0-10M as shown) followed by a further 2 hr incubation with a 25-fold excess of AN-9 (▪), or treated with AN-9 for 2 hr followed by a further 4 hr incubation with Adriamycin (□). Genomic DNA was isolated using mild conditions (Cutts et al., 2001) and subjected to gene-specific Southern hybridisation analysis. For detection of the mitochondrial genome, DNA was restriction digested with BamHI, and unreacted or intercalated drug was removed by phenol/chloroform extraction and ethanol precipitation. Samples were resuspended in 60% formamide and heat denatured at 60° C. for 5 min. Samples were resolved electrophoretically through 0.8% agarose, transferred to nylon membranes and probed with mitochondrial RNA. The percentage of double stranded DNA was calculated by phosphorimage analysis; this correlates with adduct formation in the mitochondrial genome (A and C), since the adducts behave functionally as virtual interstrand crosslinks (Zeman et al., 1998; Cullinane et al., 2000). For detection of the DHFR gene, DNA was digested with HindIII and processed as described above; however, randomly primed DHFR DNA was used as the probe for Southern analysis (B and D). Data were derived from each of two separate blots of two biological experiments, and the values are represented as the mean±SE. The results, summarised in FIG. 1, showed dramatic increases in the level of DNA adducts in the presence of AN-9, compared to Adriamycin alone where no adducts were detected. This clearly showed a direct damage mechanism which could result in the synergy displayed by the combination of Adriamycin and AN-9. It is significant that when AN-9 was added after Adriamycin, greatly enhanced levels of adducts were obtained compared to when it was added prior to Adriamycin. This provides a biochemical basis for the synergistic/antagonistic cytotoxicity relationship.

### EXAMPLE 3

### Reaction Time Dependence of AN-9 Facilitated Adriamycin Adducts

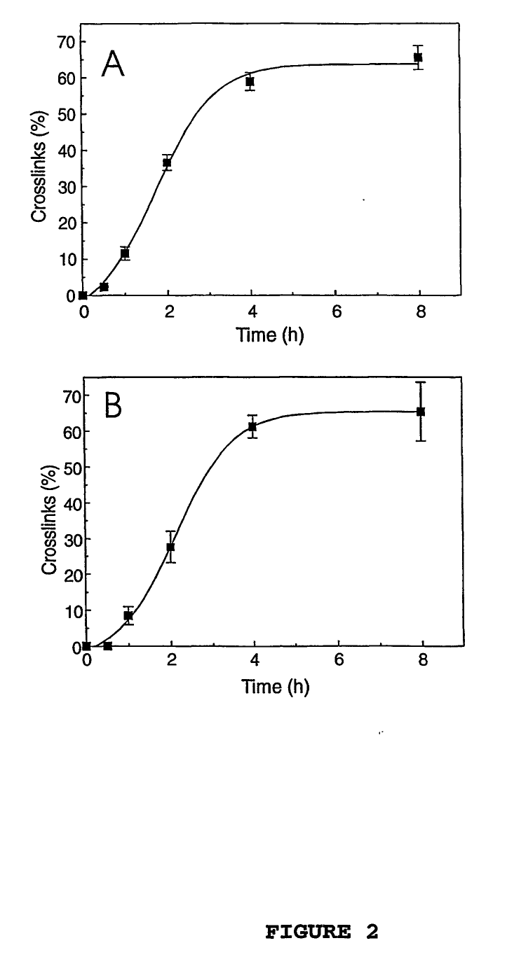

Since adducts had been demonstrated to be induced by the Adriamycin/AN-9 combination, a greater understanding of the mechanism of adduct formation was needed. It was known that the formation of Adriamycin adducts was highly time-dependent. To confirm that the adducts behave as classical Adriamycin adducts, and also to investigate the optimal conditions for adduct formation, a time course study was initiated. IMR-32 cells were treated with a combination of 4 μM Adriamycin and 100 μM AN-9 for 0-8 hr as described above. Phosphorimage analysis was used to quantitate the time-dependent formation of adducts in the mitochondrial genome (A) and DHFR gene (B). The results, presented in FIG. 2, show that adduct formation reached a plateau between 4 and 8 hr, similar to that previously shown for Adriamycin alone (Cullinane et al., 2000).

### EXAMPLE 4

### Conditions for Synergistic Adduct Formation

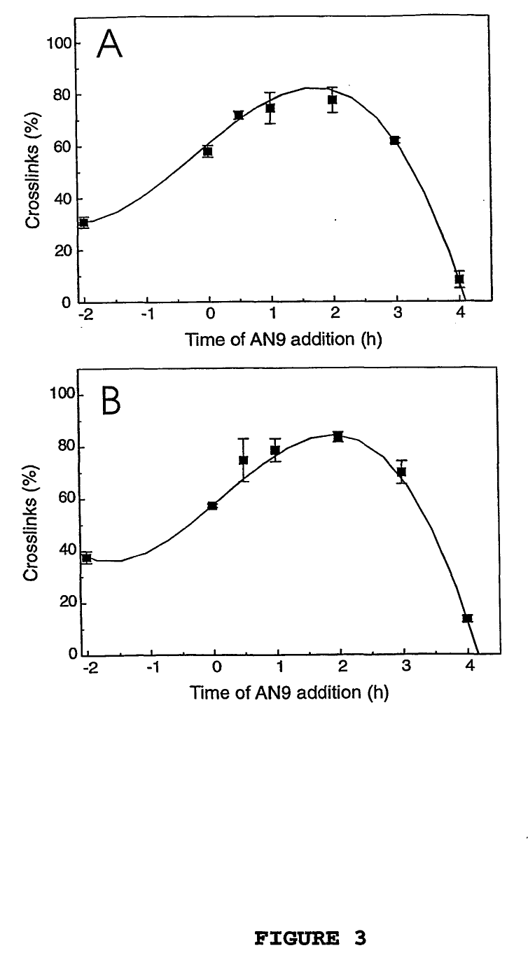

In order to gain some understanding as to why synergy was greatest when AN-9 was added at the same time as, or after, Adriamycin, a time course of addition of AN-9 was studied. IMR-32 cells were exposed to 4 μM Adriamycin for 4 hr. However, in this experiment 100 μM AN-9 was added at varying times, ranging from 2 hr prior to Adriamycin addition (designated as −2) to 2 hr after Adriamycin addition. Genomic DNA was extracted from the cells and then processed for Southern analysis. Adduct/crosslinking levels are shown in FIG. 3 for both mitochondrial (A) and nuclear (B) DNA, using probes for mtDNA and the DHFR gene respectively. Phosphorimage analysis was used to quantitate the fraction of DS DNA, a measure of adduct formation, for each treatment condition. Adduct formation fluctuated greatly, and depended on a relatively small time frame within which AN-9 was added to cells. Specifically, greatly enhanced levels of adducts were obtained when AN-9 was added shortly after Adriamycin, and this resulted in even higher adduct levels than when the drugs were added simultaneously. This enabled us to predict that if AN-9 was added shortly after Adriamycin in cytotoxicity assays, within say approximately 2 hr, then the Combination Index obtained would be even better than when the drugs were added simultaneously.

### EXAMPLE 5

### Conditions Which Antagonise Adduct Formation

To extend the analysis of the effect of the relative timing of addition of AN-9 on adduct levels, a more detailed analysis was initiated. This study was designed to include early times of addition of AN-9, so that the levels of adducts could be established where the combination exhibits antagonism as judged by cytotoxicity assays (Table 1).

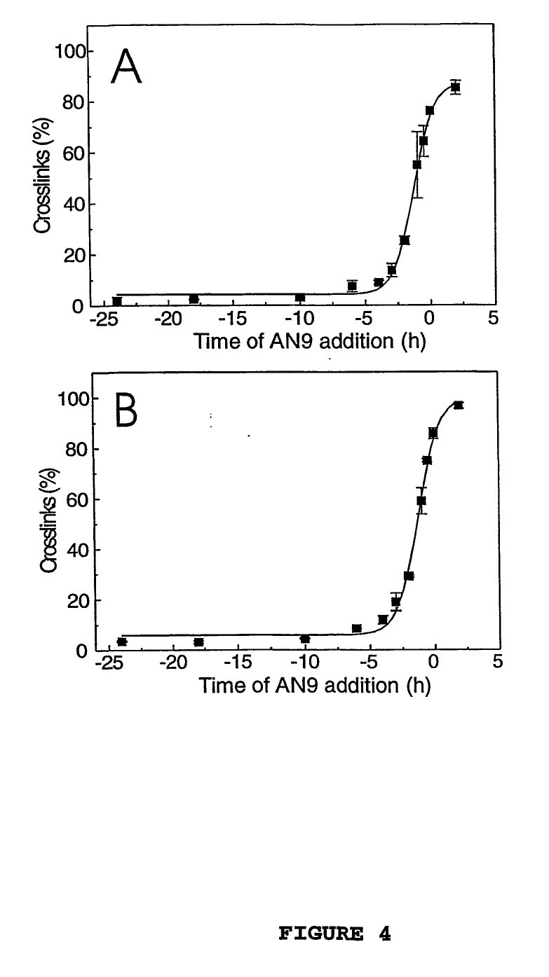

IMR-32 cells were exposed to Adriamycin (6 μm) for 4 hr, and AN-9 (125 μM) was added at varying times from 24 hr prior to Adriamycin addition (−24) to 2 hr after Adriamycin. Genomic DNA was extracted from cells and then processed for Southern analysis. Phosphorimage analysis was used for quantitation of the adducts in mtDNA (A) and the DHFR gene (B), and the results are shown in FIG. 4.

These results clearly showed that there was no enhancement of adduct levels when AN-9 was administered 5-24 hr prior to Adriamycin, but that detectable levels of adducts were observed when AN-9 was administered approximately 5 hr prior to Adriamycin, and adduct levels increased until AN-9 was administered 2 hr after Adriamycin; administration of AN-9 2 hr after Adriamycin was established in Example 4 to be optimal for adduct formation. This information enabled us to predict the best time of drug addition to obtain high levels of adducts.

### EXAMPLE 6

### Effect of AN-90N Barminomycin-Induced Crosslinks

AN-9 releases three components, pivalic acid, butyric acid (BA) and formaldehyde, when it is hydrolysed by intracellular esterases. The enhanced reaction of Adriamycin with DNA could therefore be catalysed by one or more of these components. Butyric acid released by AN-9 is likely to lead to increased adduct formation by Adriamycin, since the expression of BA causes accumulation of multi-acetylated forms of histones H3 and H4, leading to an alteration of chromatin structure (Vidali et al., 1978).

This altered chromatin structure is more sensitive to DNase I, and is a favourable configuration for transcription, and as a consequence gene regulation is altered at this level. This is accompanied by an increased accessibility to DNA by agents such as acridine orange and actinomycin D (Darzynkiewicz et al., 1969), and probably also for the intercalating agent Adriamycin. AN-9 has been shown to induce histone acetylation in HL-60 cells, presumably due to the release of BA, and this effect is transient since the basal level of acetylation is re-established 6 hr after the exposure to AN-9 (Aviram et al., 1994). We speculated that butyric acid could well lead to increased Adriamycin adduct formation, since early studies suggested that butyric acid and Adriamycin were synergistic in mouse neuroblastoma cells (Prasad, 1979), although more recent data provides little support for this notion (Kasukabe et al., 1997). Alternatively, the formaldehyde released by AN-9 could play a direct role in the increased adduct formation. Formaldehyde is one of the reagents known to lead to increased formation of Adriamycin adducts in naked plasmid and synthetic DNA, and is capable of being incorporated into the Adriamycin adduct.

In order to test whether formaldehyde or other components are important in the mechanism of enhanced adduct formation by AN-9, an anthracycline related to Adriamycin was used. This anthracycline, barminomycin, is capable of adduct formation in the absence of formaldehyde, ie it does not require activation for the formation of adducts with DNA.

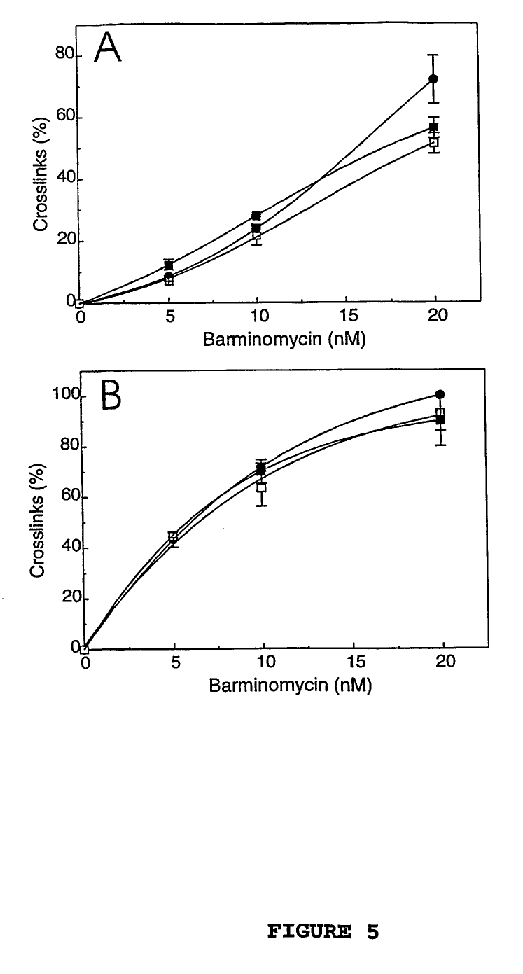

IMR-32 cells were treated with barminomycin alone (0-20 nM, ▪) for 2 hr, or barminomycin for 0.5 hr followed by a further 1.5 hr incubation with AN-9 using a 12,500-fold excess of AN-9 at each barminomycin concentration (●), or AN-9 for 2 hr followed by a further 2 hr incubation with barminomycin (□). Samples were treated as described above. Phosphorimage quantitation was used to generate results for the mitochondrial genome (A) and the DHFR gene (B). These results, illustrated in FIG. 5, demonstrated that AN-9 had no effect on the ability of barminomycin to induce DNA crosslinks, and indicate that barminomycin does not require formaldehyde to form adducts with DNA because it is essentially a “formaldehyde-activated” anthracycline (Perrin et al., 1999). This clearly distinguished barminomycin from Adriamycin, and implied that the mechanism of enhancement of formation of Adriamycin adducts by AN-9 involved activation by formaldehyde.

### EXAMPLE 7

### Incorporation of [¹⁴C] Adriamycin Into Adducts

[¹⁴C]-labelled Adriamycin was used to confirm that the adducts formed in the presence of AN-9 actually contained the Adriamycin chromophore, and also to accurately estimate the levels of adducts induced in the various treatment schedules.

IMR-32 cells were seeded into 3.5 cm petri dishes at a density of 7.5×10⁵ cells/dish 24 hr prior to exposure to 41M [¹⁴C]-Adriamycin for 4 hr. In other treatments 100 μM AN-9 was added at varying times: 2 hr prior to Adriamycin addition (−2); simultaneously (O); and 2 hr after Adriamycin (2). Cells were harvested, and the genomic DNA was isolated. Samples were then extracted twice with phenol and once with chloroform, and DNA was selectively precipitated from RNA by ammonium acetate precipitation. DNA pellets were resuspended in 100 μL TE buffer, and the concentration determined spectrophotometically at 260 nm. Aliquots of the genomic DNA (50 μL) were each added to 1 mL of Optiphase Hisafe scintillation cocktail, and the incorporation of [¹⁴C]-labelled drug into the DNA was monitored using a Wallac 1410 Liquid Scintillation Counter.

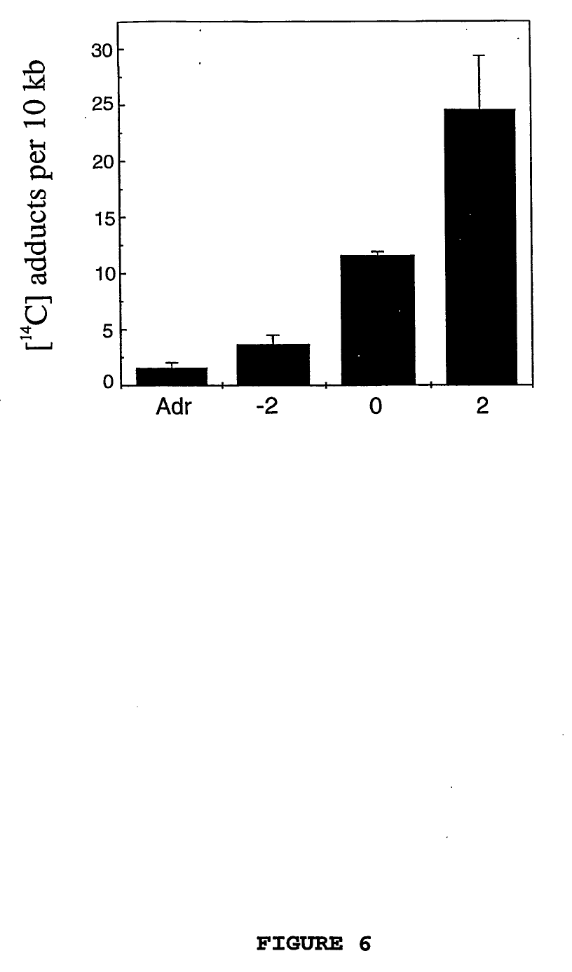

As shown in FIG. 6, the level of Adriamycin adducts in the absence of AN-9 was approximately 1.5 per 10 kb, and 3.5 per 10 kb for the 2 hr AN-9 pretreatment. When AN-9 and Adriamycin were administered simultaneously there were approximately 12 adducts per 10 kb but 24 per 10 kb when AN-9 was administered 2 hr after Adriamycin. This therefore confirmed the schedule-dependent enhancement of adducts by AN-9, and showed that the level of Adriamycin adducts could be potentiated by up to 15-fold in the presence of AN-9 under these conditions. This experiment also shows that Adriamycin alone produces low levels of adducts, even though it reportedly catalyses the cellular production of formaldehyde (Kato et al., 2000). Therefore the formaldehyde produced by Adriamycin is not sufficient to induce high levels of adducts. This may be due to a number of factors, such as inefficient subcellular localization of this pool of formaldehyde, low levels of formaldehyde production, and production of formaldehyde at inappropriate times.

### EXAMPLE 8

### [¹⁴C] Adducts Induced by AN-9 Compared to Control Compounds

It was necessary to compare the levels of adducts formed when AN-9 was added many hours prior to Adriamycin to those formed in the presence of Adriamycin alone, as it was under these conditions that the combination exhibited cytotoxic antagonism. It was also necessary to use various control compounds to further identify the components which were most responsible for enhanced adduct formation.

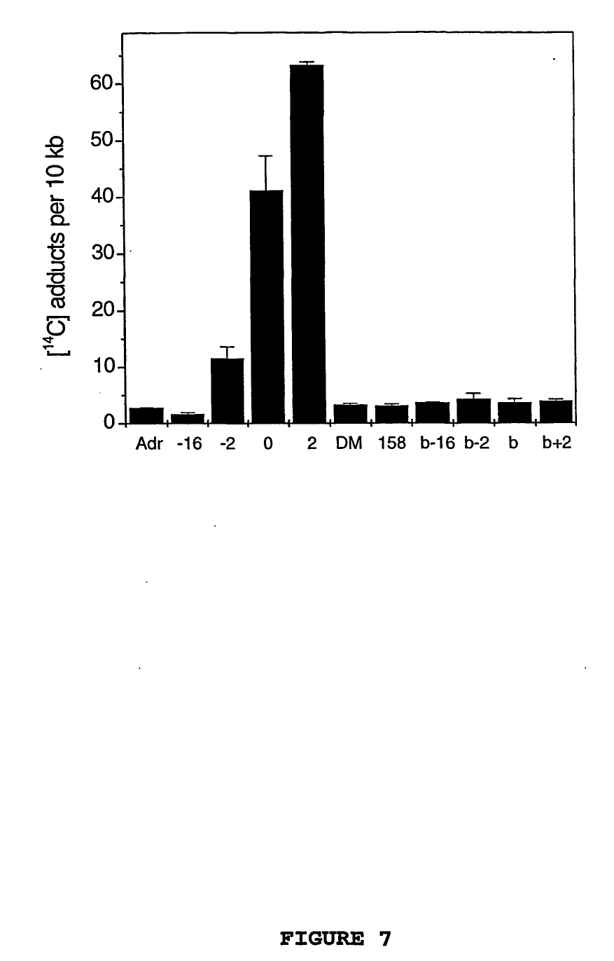

IMR-32 cells were exposed to 6 μM [¹⁴C]-Adriamycin alone (Adr) for 4 hr, or together with 125 μl AN-9 at varying times: 16 hr prior (−16), 2 hr prior (−2), simultaneously (0), and 2 hr after Adriamycin addition (2). The remaining treatments were Adriamycin with 0.5% DMSO (DM), 250 μM AN-158 (158) (which releases BA and acetaldehyde upon hydrolysis), or with sodium butyrate (1 mM) at varying times: 16 hr prior (b-16), 2 hr prior (b-2), simultaneously (b), and 2 hr after Adriamycin (b+2). Genomic DNA was extracted from the cells, and incorporation of radiolabelled drug was determined by scintillation counting to determine the level of [¹⁴C] adducts per 10 kb. The results are shown in FIG. 7.

Significantly, the levels of Adriamycin adducts were diminished when AN-9 was added 16 hr prior to Adriamycin (1.7/10 kb) compared to using Adriamycin alone (2.8/10 kb). This represented a 40% loss of adducts, therefore explaining why the AN-9 treatment under these conditions would have been less effective. In contrast, there was an approximately 20-fold enhancement compared to Adriamycin alone when AN-9 was added 2 hr after Adriamycin. The control results showed that the DMSO vehicle in which AN-9 was dissolved did not contribute to adduct formation. Butyric acid was used as a control under various conditions to test directly whether adduct formation could be increased by the exposure of cells to this agent. However, there were no significant increases in adduct levels under any of the treatment conditions.

### EXAMPLE 9

### [¹⁴C] Adduct Formation Induced by a Series of Aldehyde-Releasing Compounds Related to AN-9

In order to test whether formaldehyde-releasing drugs other than AN-9 were efficient at facilitating Adriamycin adducts, a series of aldehyde-releasing compounds was assessed. The structures of these aldehyde-releasing compounds and the hydrolysis products which they release are summarised in Table 2.

TABLE 2

| | | | | NMR(CDCl₃) ppm |
|---|---|---|---|---|
| COMPOUNDS | NAME | STRUCTURE | PRODUCTS | Spectra |
| AN-9 | Pivaloyloxymethyl Butyrate | 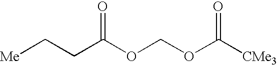 | 1) Butyric Acid 2) Formaldehyde 3) Pivalic Acid | 0.92(t, MeCH₂, 3H), 1.16(s, t-Bu, 9H), 1.45 (d, MeCH, 3H), 1.67(sext, MeCH₂, 2H), 2.3 (t, CH₂CO, 2H), 6.86(q, OCH₂O). |
| AN-1 | Butyroyloxymethyl Butyrate | 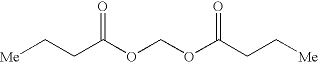 | 1) 2 eq Butyric Acid 2) Formaldehyde | 0.95(t, Me, 3H), 1.63(sext, CH₂Me, 4H), 2.33 (t, CH₂CO, 4H), 5.78(s, OCH₂O, 2H). |
| AN-11 | Ethylidene Dibutyrate | 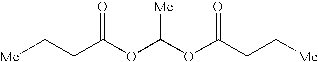 | 1) 2 eq Butyric Acid 2) Acetaldehyde | 0.95(t, Me, 3H), 1.47(d, MeCH, 3H), 1.65 (sext, CH₂Me, 4H), 2.3(t, CH₂CO, 4H), 6.66 (q, CH, 1H). |
| AN-7 | Butyroyloxymethyldiethyl Phosphate | 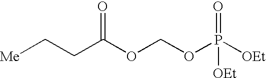 | 1) Butyric Acid 2) Formaldehyde 3) Phosphoric Acid 4) Ethanol | 5.63(d, 2H, OCH₂O), 4.13(d quint, 4H, two CH₂OP), 2.36(t, 2H, COCH₂), 1.69 (sext, 2H, CH₂CH₂CO), 1.34 (td, 6H, two MeCH₂O), 0.96 (t, 3H, Me). |
| AN-88 | 1-Butyroyloxyethyldiethyl Phosphate | 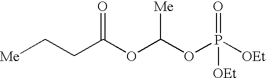 | 1) Butyric Acid 2) Acetaldehyde 3) Phosphoric Acid 4) Ethanol | 6.47(dq, 1H, CHMe), 4.08 (ddquint, 4H, two CH₂OP), 2.28 (t, 2H, COCH₂), 1.62(sext, 2H, CH₂CH₂CO), 1.49 (d, 3H, CHMe), 1.29(tdd, 6H, two MeCH₂O), 0.91(t, 3H, Me) |
| AN-158 | 1-Pivaloyloxyethyl Butyrate | 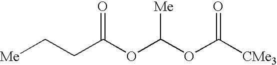 | 1) Butyric Acid 2) Acetaldehyde 3) Pivalic Acid | 0.95(t, MeCH₂, 3H), 1.2(s, t-Bu, 9H), 1.45 (d, MeCH, 3H), 1.63(sext, CH₂Me, 2H), 2.3 (t, CH₂CO), 6.84 (q, CH, 1H). |
| AN-184 | 1-Propionylyloxyethyl Pivalate | 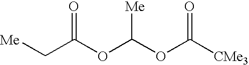 | 1) Propionic Acid 2) Acetaldehyde 3) Pivalic Acid | 1.12(t, Me, 3H), 1.2(s, CMe₃, 9H), 1.45 (d, Me, 1), 2.32 (q, CH₂CO, 2H), 6.85(q, OCH₂O, 1H). |
| AN-185 | 1-Isobutyroylyloxyethyl Pivalate | 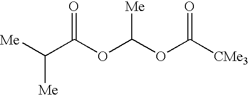 | 1) Isobutyric Acid 2) Acetaldehyde 3) Pivalic Acid | 1.2(d, Me¹, 3H), 1.21(d, Me², 3H), 1.23 (s, CMe₃, 9H), 1.5(d, MeCH, 3H), 2.54(sept, CHCO₂, 1H), 6.83 (q, CH, 1H). |
| AN-36 | Propionyloxymethyl Pivalate | 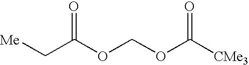 | 1) Propionic Acid 2) Formaldehyde 3) Pivalic Acid | 1.15(t, Me, 3H), 1.2(s, t-Bu, 9H), 2.37 (q, CH₂CO₂, 2H), 5.76(s, OCH₂O, 2H). |
| AN-38 | Valeroyloxymethyl Pivalate | 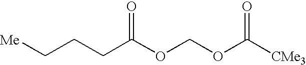 | 1) Pentanoic Acid 2) Formaldehyde 3) Pivalic Acid | 0.88(t, Me, 3H), 1.22(s, t-Bu, 9H), 1.35 (sext, CH₂Me, 2H), 1.62 (quint, CH₂CH₂Me, 2H), 2.33(t, CH₂CO₂, 2H), 5.73(s, OCH₂O, 2H) |
| AN-37 | Isobutyroyloxymethyl Pivalate | 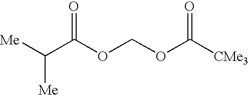 | 1) Isobutyric Acid 2) Formaldehyde 3) Pivalic Acid | 1.18(d, Me, 6H), 1.22(s, t-Bu, 9H), 2.6 (sept, CH, 1H), 5.77(s, OCH₂O, 2H). |
| AN-188 | Ethylidene Dipropionate | 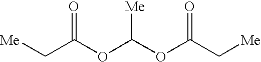 | 1) Propionic Acid 2) Acetaldehyde 3) Propionic Acid | 1.15(t, Me, 3H), 1.47(d, MeCH, 3H), 2.3 (t, CH₂CO, 4H), 6.66(q, CH, 1H). |
| AN-190 | Oxalic acid bis-(2,2-dimethylpropionyloxymethyl)ester | 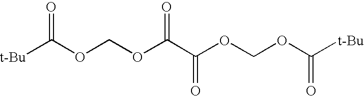 | 1) 2 eq Pivalic Acid 2) Oxalic Acid 3) 2 eq Formaldehyde | 1.22(s, 9H, t-Bu), 5.7(s, 2H, CH₂). |
| AN-192 | Succinic acid bis-(2-2-dimethylpropionyloxymethyl)ester | 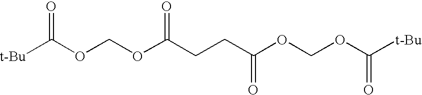 | 4) 2 eq Pivalic Acid 5) Succinic Acid 6) 2 eq Formaldehyde | 1.23(s, t-Bu, 18H), 2.68(s, CH₂CH₂, 4H), 5.75(s, OCH₂O, 4H). |
| AN-193 | Succinic acid dibutyryloxymethyl ester | 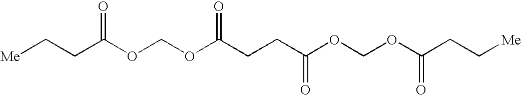 | 7) 2 eq Butyric Acid 8) Succinic Acid 9) 2 eq Formaldehyde | 0.95(t, Me, 9H), 1.64 (sext, MeCH₂, 4H), 2.34(t, CH₂CO, 4H), 2.67 (s, CH₂CH₂, 4H), 5.76(s, OCH₂O, 4H). |
| AN-194 | Oxalic acid dibutyryloxymethyl ester | 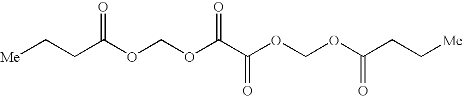 | 10) 2 eq Butyric Acid 11) Oxalic Acid 12) 2 eq Formaldehyde | 0.96(t, Me, 3H), 1.7(sext, CH₂Me, 2H), 2.38 (t, CH₂CO, 2H), 5.92(s, OCH₂O, 2H). |
| AN-189 | Oxalic acid bis-(1-butyryloxy-ethyl)ester | 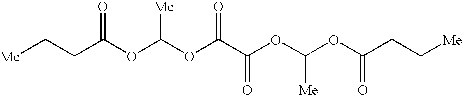 | 1) 2 eq Butyric Acid 2) Oxalic Acid 3) 2 eq Acetaldehyde | 0.96(t, Me, 3H), 1.58(d, MeCH, 3H), 1.65 (sext, CH₂, 2H), 2.34(t, CH₂, 2H), 6.95(qd, CH, 1H). |
| AN-191 | Succinic acid bis-(1-butyryloxy-ethyl)ester | 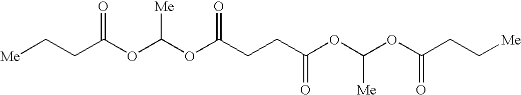 | 1) 2 eq Butyric Acid 2) Succinic Acid 3) 2 eq Acetaldehyde | 0.92(t, Me, 6H), 1.43(d, Me, 6H), 1.6 (sext, CH₂, 4H), 2.3(t, CH₂, 4H), 2.6(s, CH₂CH₂, 4H), 6.8 (q, OCHO, 2H). |

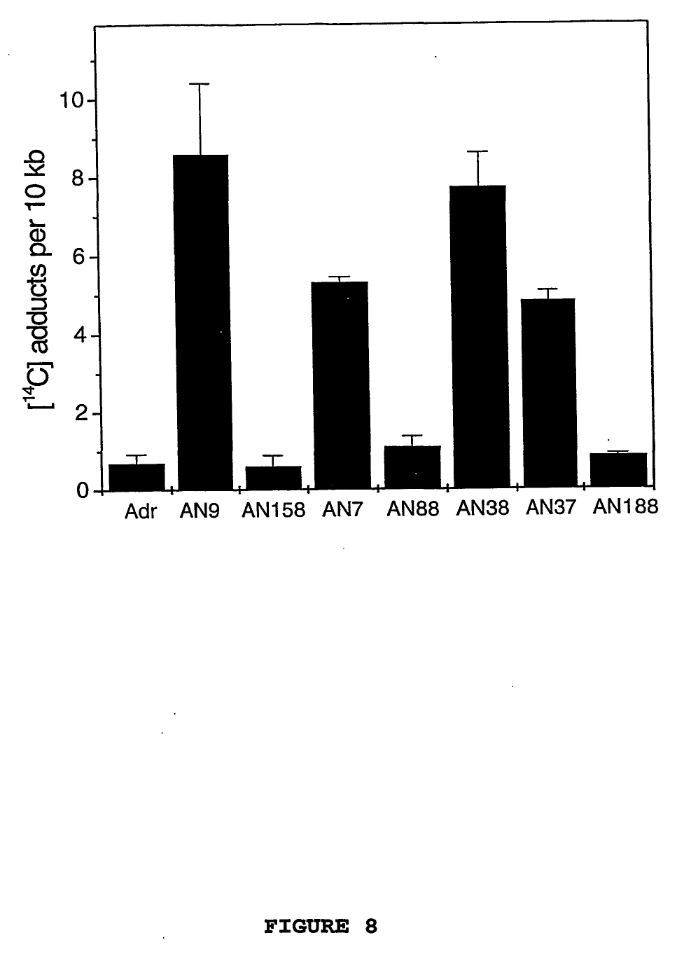

There was a possibility that only AN-9 possessed the unique ability to contribute to adduct formation. This would be apparent if intact AN-9, ie. AN-9 prior to the esterase-dependent release of products, facilitated crosslink formation. It was also possible that no other compound was as efficient as AN-9 at releasing the formaldehyde needed for adduct formation. Therefore, IMR-32 cells were treated with 4 μM [¹⁴C] Adriamycin and were simultaneously treated with 100 μM of selected aldehyde-releasing compound. Cells were harvested, genomic DNA was extracted and scintillation analyses were performed as described above. The results are shown in FIG. 8.

Significantly, only the formaldehyde-releasing drugs AN-9, AN-7, AN-37, and AN-38 enhanced adduct levels compared to levels obtained in the presence of Adriamycin (ADR) alone. However, the enhancement varied dramatically depending on the aldehyde-releasing compound employed.

AN-9 and AN-38 were the most effective compounds, and exhibited equivalent levels of activation. They are very similar in structure, and therefore may localize similarly in subcellular compartments, and be hydrolysed to constitutive products with similar efficiency. AN-7 and AN-37 also exhibited good activity, but were not as effective as AN-9 and AN-38. This could be due to one or more of a number of factors, such as

1) different rates of hydrolysis;

2) differences in subcellular localization and/or cell uptake; and

3) interference with adduct formation by other hydrolysis products such as isobutyric acid.

When the control aldehyde-releasing compound AN-158 was used, there was no enhancement of adduct formation. This drug is almost identical to AN-9, in that it releases butyric acid and pivalic acid; however, it releases acetaldehyde rather than formaldehyde. These data therefore provide clear evidence that the formaldehyde component of AN-9 contributed significantly to adduct formation.

Other control acetaldehyde-releasing prodrugs which were used (AN-88 and AN-188) also reinforced the concept that it was the released formaldehyde which was solely responsible for increased adduct formation.

### EXAMPLE 10

### [¹⁴C] Adriamycin Adducts Obtained with Increasing Concentrations of AN-9

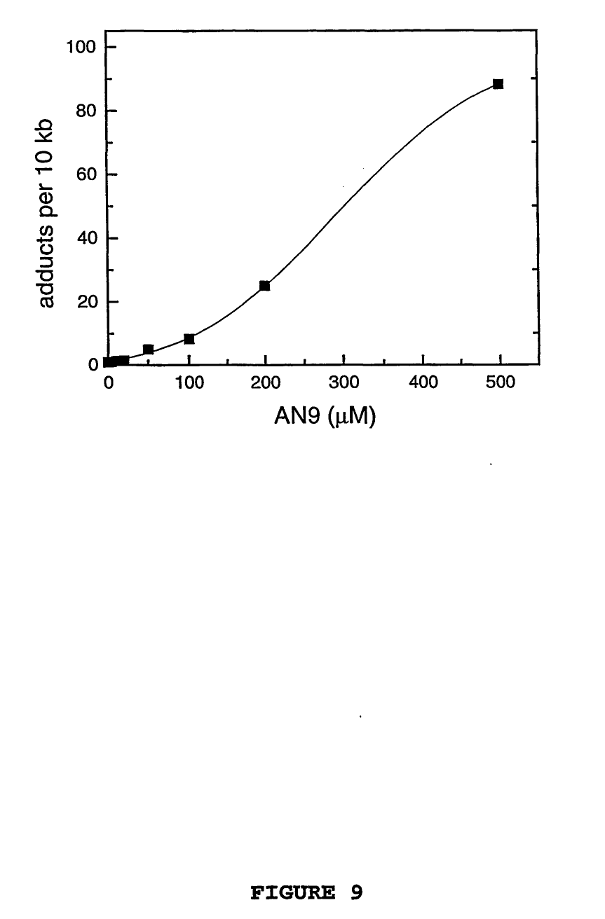

To test whether a greater fraction of Adriamycin can be utilized for adduct formation when the amount of available formaldehyde is increased, the concentration dependence of AN-9 was investigated. IMR-32 cells were treated with [¹⁴C] Adriamycin at a constant concentration of 4 μM. AN-9 was added 2 hr after Adriamycin at concentrations ranging from 4 μM (1:1) to 500 μM (1:125), and the treatment continued for a further 2 hr. Cells were harvested, DNA was extracted and samples were analysed for incorporation of [¹⁴C]. The results are shown in FIG. 9.

At low levels of Adriamycin:AN9, (1:1-1:5), the enhancement of adduct formation was poor, but at higher concentrations there was a near linear relationship between AN-9 concentration and Adriamycin adducts. Significantly, the ratio of Adriamycin:AN-9 routinely employed in experiments is 1:20-1:25, and in this experiment yielded 10 adducts per 10 kb. However, at 500 μM AN-9 (1:125) adduct levels were approximately 10 fold higher. This represents an approximately 100-fold potentiation of Adriamycin adduct levels in the presence of AN-9.

It is therefore evident that by modulating the level of AN-9, the level of adducts can be enhanced by high aldehyde-releasing compound:chemotherapeutic agent ratios. This is a particularly significant finding, as it implies that very low concentrations of Adriamycin can be employed for adduct formation if high levels of formaldehyde are available. At 500 μM AN-9, approximately 5% of the total Adriamycin added to cells was incorporated into DNA adducts. Since saturation (ie, a plateau) was not observed, this percentage can probably be expected to be further enhanced. Furthermore, since Adriamycin adducts are unstable, with a half-life of (Phillips and Cullinane, 1999) 5-40 hr, the actual level of adducts in the undisturbed cellular environment is presumed to be higher than quantitated in these experiments.

### EXAMPLE 11

### Reversal of Formaldehyde-Mediated Effects by Semi-Carbazide

Since formaldehyde has been shown to be a critical element in the formation of DNA adducts, it was important to establish if these adducts were responsible for the enhanced cytotoxicity displayed by the Adriamycin/AN-9 combination. Formaldehyde was sequestered by the addition of high concentrations of semi-carbazide (sc). The results of this study are shown in Table 3.

TABLE 3

| | Adducts | Relative |
|---|---|---|
| Treatment | (lesions/10 kb) | survival |
| Adriamycin | 0.7 ± 0.01 | 1.00 ± 0.12 |
| Adriamycin + AN-9 | 11.0 ± 0.5 | 0.35 ± 0.06 |
| Adriamycin + AN-9 + sc (250 μM) | 6.2 ± 1.1 | 0.37 ± 0.11 |
| | (6.7 ± 0.6) | |
| Adriamycin + AN-9 + sc (500 μM) | 4.9 ± 1.0 | 0.71 ± 0.08 |
| | (4.6 ± 0.2) | |
| Adriamycin + AN-9 + sc (1 mM) | 3.9 ± 0.3 | 0.88 ± 0.22 |
| | (3.2 ± 0.2) | |
| Adriamycin + sc (1 mM) | 0.7 ± 0.01 | 1.2 ± 0.16 |

Adducts (lesions/10 kb) were determined by incubating IMR-32 cells in the presence of 2 μM [¹⁴C]Adriamycin for 2 hr. followed by an additional 2 hr in the presence or absence of 100 μM AN-9. Semi-carbazide addition was at the same time as Adriamycin (ie 2 hr before AN-9). However, values in parentheses indicate that semi-carbazide addition was at the same time as AN-9 treatment (ie 2 hr after Adriamycin). As expected, the level of DNA adducts was dramatically reduced by incubation of cells with increasing ratios of semi-carbazide. Little difference was observed between adding semi-carbazide at the same time as Adriamycin or at the same time as AN-9 (2 hr later).

Cell viability assays were used to measure the effect of semi-carbazide inclusion on the interaction displayed by the Adriamycin/AN-9 combination. To obtain data representing relative survival, cells were seeded at a density of 1×10⁴ per well and exposed to increasing concentrations of Adriamycin in the presence or absence of 50 μM AN-9 and/or semi-carbazide (0.25, 0.5 or 1.0 mM as indicated) for 72 hr. IC₅₀ values for various combinations are shown as relative survival compared to Adriamycin alone. Incubation of Adriamycin and AN-9 with increasing concentrations of semi-carbazide resulted in increasing levels of protection from the drug combination and at the highest semi-carbazide concentration (1 mM) cell viability was similar to that displayed by Adriamycin alone. Overall, the critical role of formaldehyde was confirmed by reversal of formaldehyde-mediated effects by semi-carbazide, which reduced adduct formation and also abolished the cytotoxicity resulting from the interaction of AN-9 with Adriamycin. It is therefore clear that the formation of adducts is at least in part associated with (or responsible for) the synergy displayed by the AN-9/Adriamycin combination.

### EXAMPLE 12

### The Ability of Hexamethylenetetramine to Facilitate Adriamycin Adducts

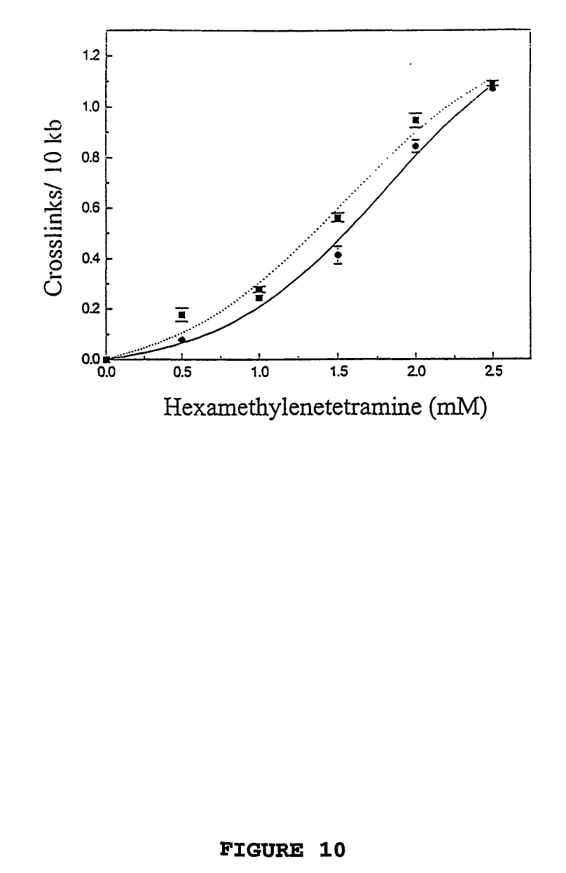

IMR-32 (1×10⁶) cells were seeded into 10 cm petri dishes and allowed to attach overnight. Cells were then treated with 15 μM Adriamycin and 4 hr later with 0-2.5 mM hexamethylenetetramine (see structure below). Cells were harvested after 8 hr and the DNA extracted using a modified procedure of a QIAamp DNA Blood Mini Kit (QIAGEN), restriction digested and separated electrophoretically on a 0.5% agarose gel (Cutts et al, 2001). The gel was transferred to a nitrocellulose membrane and Southern hybridisation was used to indicate the nuclear DHFR gene and the mitochondrial genome. The virtual crosslinks were calculated as lesions per 10 kb and are shown in FIG. 10 at each hexamethylenetetramine concentration for nuclear DNA (▪) and mt DNA(●). Virtual crosslinks were not detected with Adriamycin treatment or hexamethylenetetramine treatment as single compounds. The overall levels of lesions are approximately 60-fold lower than that detected by the [¹⁴C] binding assay, due mainly to the loss of adducts using the harsher Southern hybridisation technique (Cutts et al., 2001). These results demonstrate that formaldehyde-releasing compounds (other than the specific aldehyde-releasing compounds examined) have the ability to facilitate adduct formation. However, hexamethylenetetramine is not as effective as AN-9 and similar aldehyde-releasing compounds and this is probably due to the slow release of formaldehyde by hexamethylenetetramine which is favoured under conditions of low pH, as opposed to the rapid hydrolysis of aldehyde-releasing compounds by esterases.

Structure of Hexamethylenetetramine

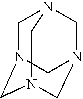

### EXAMPLE 13

### Stability of AN-9 Induced Adriamycin Adducts in Cells

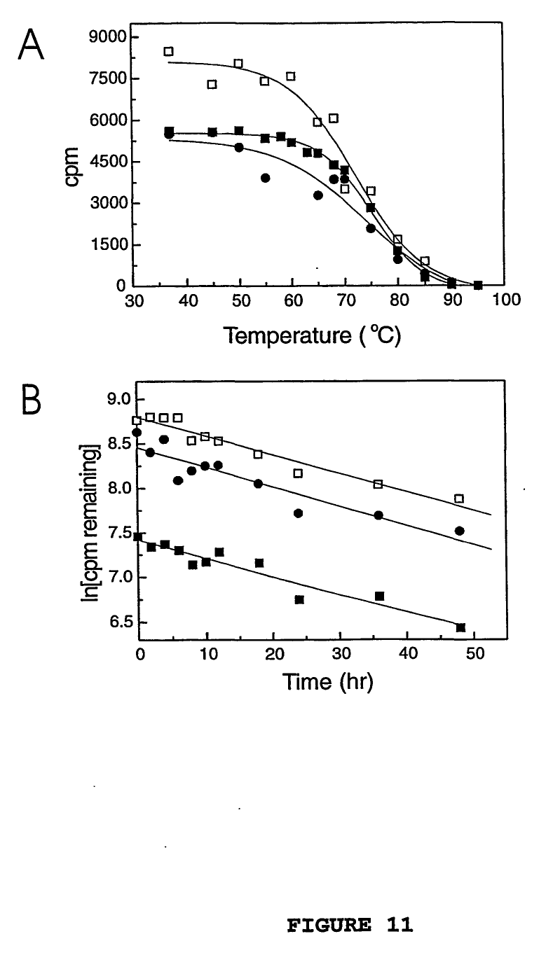

In order to determine the stability of AN-9 induced DNA adducts we performed the following experiment. We seeded IMR-32 cells at a density of 2.5×10⁶ cells per 10 cm petri dish. The following day cells were pretreated with 4 μM [¹⁴C] Adriamycin for 2 hr and then treated with 260 μM AN-9 for a further 2 hr. Cells were harvested and genomic DNA was isolated using a modified QIAamp procedure. Residual intercalated Adriamycin was removed by two phenol extractions, one chloroform extraction and ethanol precipitation. Samples were exposed to various times of standing at 37° C. or varying temperatures, and unbound drug was removed by a further phenol/chloroform extraction. Residual [¹⁴C] Adriamycin-DNA adducts were assessed by scintillation counting. To confirm that the DNA adducts induced in cells by AN-9 were the same as those produced in vitro, [¹⁴C] Adriamycin was also used to form adducts under different conditions in cell free systems. This allowed the fate of the Adriamycin chromophore to be assessed in response to elevated temperature and extended times at 37° C. The results of this study are shown in FIG. 11. Specifically, the conditions for formation of adducts were formaldehyde-facilitated adducts in vitro (1), AN-9/esterase facilitated adducts in vitro (!) and Adriamycin/AN-9 induced adducts in cells (+). Adducts purified from the three different environments all showed similar temperature lability and adduct-DNA dissociation rates, indicating that the adducts in cells are most likely of identical structure to those produced in cell free systems. Specifically, the melting temperature (Tm) of formaldehyde-mediated adducts in vitro was 72.2±1.6° C., with a half life at 37° C. of 33.2±2.7 hr. AN-9/esterase-mediated adducts in vitro had a Tm of 74.3±3.5° C. and a half-life of 31.7±4.4 hr while Adriamycin adducts formed in cells in the presence of AN-9 had a Tm of 75.1±0.4° C. and a half-life of 34.0±3.0 hr.

### EXAMPLE 14

### Sequence Specificity of AN-9 Induced Adriamycin-DNA Adducts in Cells

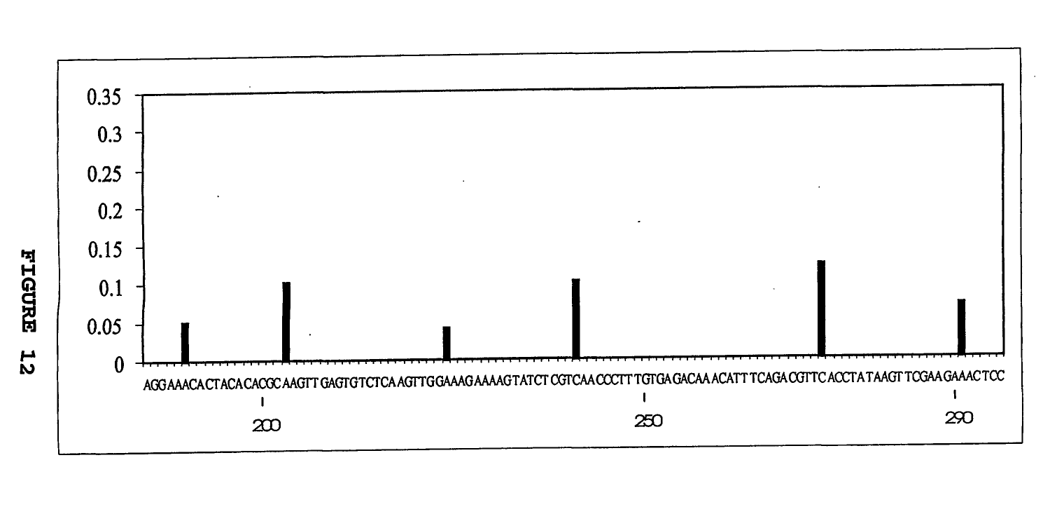

IMR-32 cells were seeded at a density of 1.5×10⁶ cells per 10 cm petri dish and allowed to attach overnight. Cells were treated with 4 μM Adriamycin and 500 μM AN-9 for a total of 4 hr (the AN-9 was added 2 hr after Adriamycin) then harvested and washed twice with PBS. Total genomic DNA was extracted using a modified QIAamp procedure. Genomic DNA was restriction digested with EcoRI to isolate a 340 bp alpha satellite repeat. DNA fragments were 3′ end-labelled with [³²P]DATP using the Klenow fragment of DNA polymerase and then restriction digested using HaeIII. The 296 bp band was isolated and digested at 37° C. for 1.5 hr using λ exonuclease to progressively release 5′ nucleotides. Adducts provide direct blockages to this stepwise digestion. The remaining lengths of radiolabelled fragment represent blockages and therefore reveal adduct binding sites. The sequence specificity of blockages produced by the Adriamycin/AN9 combination in a region of this sequence is shown in FIG. 12. The sequence is presented 3′-5′. Bands were resolved by electrophoresis and quantitated by PhophorImage analysis to determine the relative intensity of each band corresponding to a drug blockage site. Each blockage site is represented as the mole fraction of total blockages at the corresponding sequence. The striking 5′-GC specificity of the combination is similar to that observed in numerous in vitro experiments, indicating that the adducts formed in vitro and in cells are likely to be of the same structure. It is apparent that the adducts occur at every 3′-CG sequence examined. Of the 6 blockages represented, this includes all four of the 5′-GC sites represented and two 5′-GG sites within the fragment that were analysed. The blockage to exonuclease digestion generally occurs anywhere from 1-4 nucleotides prior to the site of adduct formation, however at the last site the blockage is 4-6 nucleotides prior to the likely site of adduct formation, and this may be due to structural deviations posed to λ exonuclease at the extreme end of the fragment.

### EXAMPLE 15

### Binding of AN-9 Induced Adriamycin Adducts to RNA, DNA and Protein

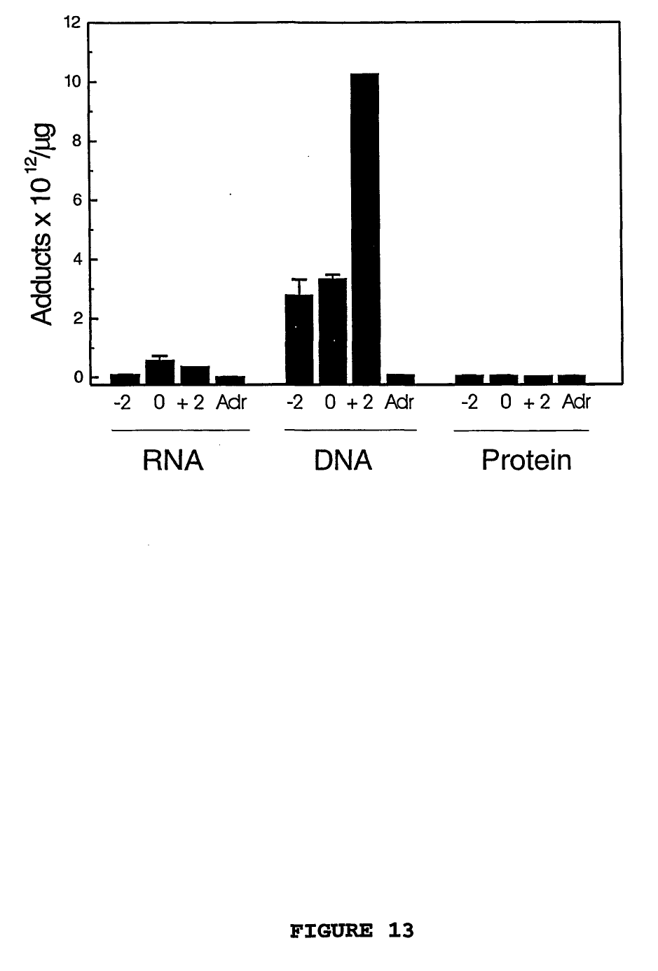

In order to assess whether AN-9 causes Adriamycin adduct formation with cellular macromolecules other than DNA, adduct formation in RNA, DNA and protein were assessed simultaneously. IMR-32 cells (1×10⁶ cells) were seeded into 3.5 cm petri dishes and cells were allowed to attach overnight. Cells were treated with 6 μM [¹⁴C]-Adriamycin for a total of 4 hr and AN-9 was used at a final concentration of 300 μM. Treatments consisted of Adriamycin alone (Adr), AN-9 added 2 hr earlier (−2), AN-9 added simultaneously (0), or AN-9 added 2 hr after Adriamycin (+2). Cells were then harvested and RNA, DNA and protein were isolated simultaneously using TRI Reagent (Astral). Incorporation of radioactive Adriamycin into each of the fractions was assessed by scintillation counting. The concentration of RNA, DNA and protein in each fraction were also determined. FIG. 13 shows the number of adducts per pg of nucleic acid or protein. This figure demonstrates that adducts mainly form within DNA. DNA was also the dominant target for adduct formation with mitoxantrone and AN-9 (data not shown).

### EXAMPLE 16

### Alternative Aldehyde-Releasing Compounds

The series of aldehyde-releasing compounds that were examined was extended to include those that release two molecules of formaldehyde per aldehyde-releasing compound and more than one molecule of butyric acid per aldehyde-releasing compound (compounds AN-189 to AN-194). These compounds were synthesised using the same procedure used in the synthesis of AN-9 as described in A. Nudelman, M. Ruse, A. Aviram, R. Rabizadeh, M. Shaklai, Y. Zimrah and A. Rephaeli, 1992. The procedure was modified by replacing butyric acid with another acid and chloromethyl pivalate with the subject chloromethyl ester.

To examine adduct formation in the presence of these aldehyde-releasing compound, IMR-32 cells were seeded into 3.5 cm petri dishes at a density of 7.5×10⁵ cells/dish 24 hr prior to exposure to 2 μM [¹⁴C] Adriamycin for 4 hr. These were simultaneously incubated with the indicated aldehyde-releasing compound at a final concentration of 100 μM. Samples were harvested as indicated previously and DNA was assessed for incorporation of radioactively labelled Adriamycin. The results are shown below in Table 4.

TABLE 4

| Treatment | Adducts/10 kb |
|---|---|
| Adr alone | 0.54 ± 0.04 |
| Adr + AN-9 | 4.63 ± 0.59 |
| Adr + AN-1 | 11.45 ± 0.65 |
| Adr + AN-38 | 5.13 ± 1.42 |
| Adr + AN-37 | 3.26 ± 0.26 |
| Adr + AN-188 | 0.7 ± 0.16 |
| Adr + AN-189 | 0.5 ± 0.04 |
| Adr + AN-191 | 0.52 ± 0.01 |
| Adr + AN-192 | 27.1 ± 0.8 |
| Adr + AN-194 | 29.5 ± 1.5 |

At this concentration of Adriamycin (Adr), background levels of adducts were observed with the use of Adriamycin alone. This figure did not increase significantly when acetaldehyde-releasing drugs were used (AN-188, AN-189, AN-191). While AN-189 and AN-191 each released two molecules of acetaldehyde rather than the one released by AN-188, this did not enhance adduct formation. Interestingly, the derivative that releases two molecules of butyric acid and one of formaldehyde enhanced adduct formation at least two-fold higher than AN-9. This indicates an added benefit of this small structural change. The reason for enhanced adduct formation may be better localisation of this drug to the nucleus and/or enhancement of formaldehyde-facilitated adducts by butyric acid. The use of the new aldehyde-releasing compounds which each release two molecules of formaldehyde greatly improved Adriamycin adduct formation with at least a 5-fold increase of adducts compared to the same concentration of AN-9. This increase is far greater than the expected 2-fold increase that was expected to flow from the release of two molecules of formaldehyde instead of one.

### EXAMPLE 17

### Compounds that Release Aldehyde and an Agent that Enhances Intracellular Levels of Aldehyde

The results provided above indicated that compounds that release an agent that enhances intracellular levels of aldehyde would be good candidates for use in the methods of the invention. One particularly preferred compound of this class releasing BSO formaldehyde and acid in situ. This compound is synthesised using the same procedure used in the synthesis of AN-9 as described in Nudelman, 1992. The procedure is modified by replacing butyric acid with BSO [Me(CH₂)₃—S(═O) (═NH)—CH₂—CH₂CH(—NH₂)—COOH] and replacing chloromethyl pivalate with chloromethyl butyrate. The ═NH and —NH₂ functional groups of BSO are firstly protected with a protecting group such as t-butoxycarbonyl (t-BOC) before being reached with chloromethyl butyrate. In a final step, the protecting groups are removed with an acid.

### EXAMPLE 18

### Useable Chemotherapeutics Agents

In order to test whether chemotherapeutic agents other than the anthracyclines can be activated to form DNA adducts by AN-9, cells were treated with the anthracenedione drug mitoxantrone in the presence and absence of AN-9. Mitoxantrone (see structure below) has been shown to form adducts in the presence of formaldehyde in cell-free systems (Parker et al., 1999). IMR-32 cells (1×10⁶) were seeded into 3.5 cm petri dishes and allowed to attach overnight. Cells were incubated with 20 μM [¹⁴C] mitoxantrone (Mitox) and 500 μM AN-9 at times ranging from 16 hr before mitoxantrone addition to 4 hr after (−16 to +4). Total mitoxantrone incubation time was 6 hr. Cells were harvested and DNA extracted using a modified QIAamp procedure prior to [¹⁴C] mitoxantrone counts and DNA concentration being determined. The adducts detected per 10 kb are shown below in Table 5.

Structure of Mitoxantrone

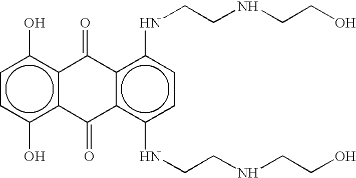

TABLE 5

| Treatment | Time of AN-9 addition | Adducts/10 kb |
|---|---|---|
| Mitox alone | | 0.36 ± 0.06 |
| Mitox + AN-9 | −16 | 0.77 ± 0.01 |
| Mitox + AN-9 | −2 | 0.93 ± 0.04 |
| Mitox + AN-9 | simultaneous | 1.4 ± 0.12 |
| Mitox + AN-9 | +2 | 1.62 ± 0.01 |
| Mitox + AN-9 | +4 | 1.78 ± 0.1 |

This result shows that like Adriamycin, mitoxantrone can also be activated to form adducts in the presence of AN-9 in a cell culture assay. Furthermore, the pattern of activation is similar to Adriamycin in that the adducts are more pronounced when AN-9 is added after-mitoxantrone.

### EXAMPLE 19

### In Vito Efficacy

In order to determine the efficacy of the compounds of the invention human prostate carcinoma (PC-3) or human uterine sarcoma (MES-SA/DX5, resistant to doxorubicin) cells (5×10⁶) are implanted subcutaneously into both rear flanks of athymic nude mice (nu/nu, weighing 17-20 g). After 2-3 weeks, randomized groups of animals, each containing 10 tumour-bearing mice, are treated as follows:

(a) Control group, vehicle only, weekly×2

(b) Doxorubicin as single agents 2 mg/kg, weekly×2

(c) Doxorubicin as single agent, 4 mg/kg, weekly×2

(d) Doxorubicin as single agent, 8 mg/kg, weekly×2

(e) Test compounds of formula I at 20 mg/kg, weekly×2

(f) Test compounds of formula I at 40 mg/kg, weekly×2

(g) Test compounds of formula I at 80 mg/kg, weekly×2

(h) The combination of treatments b and e

(i) The combination of treatments c and f

In the combination treatment, doxorubicin is given first and the compound is given 4 hr later.

When control tumours reach the size of 2 g, the experiment is terminated, mice are weighed twice weekly, and tumour measurements are taken by calipers twice weekly. Tumour measurements are converted to tumour weight by the formula L²×W/2 where L and W are the length and width of the tumour respectively. These calculated tumour weights are used to determine the termination date. Upon termination, all mice are weighed, sacrificed, and their tumours excised. Tumours are weighed, and the mean tumour weight per group is calculated. In this model, the mean control tumour weight/mean treated tumour weight×100% (C/T) is subtracted from 100% to give the tumour growth inhibition (TGI) for each group. Some animals experience tumour shrinkage. For these mice, the final weight of a given tumour is subtracted from its own weight at the start of treatment. The difference divided by the initial tumour weight is the % shrinkage. Mean % tumour shrinkage is calculated from data in the group that experienced regressions.

### EXAMPLE 20

### In Vivo Adduct Formation

Two animals from each of the treatment groups defined in Example 19 are sacrificed 30 min after the last treatment. Tumour, heart, liver, kidney, and brain are isolated and snap frozen. To determine the conditions (eg, best test compound, best schedules) which lead to adduct formation in tumour DNA samples, a Southern hybridisation-based assay is used. Human tumour and mouse tissue samples are maintained at −80° until they are processed for DNA isolation. DNA isolation procedures are designed to minimise loss of Adriamycin adducts. Specifically, frozen samples are homogenised mechanically in frozen homogenisers. DNA is isolated from the frozen powder using a modified QIAamp procedure, and processed as previously described for the Southern hybridisation procedure (see Example 2). However, as the previously described hybridisation procedure is only specific for human samples (human DHFR and mitochondrial DNA probes) the DNA extracted from mouse tissues will be restriction digested with Kpn I (for DHFR detection), or an enzyme which linearises the mouse mitochondrial genome (eg. Xho I) for detection of adducts in mitochondrial DNA. These samples are subjected to hybridisation with mouse DRFR and mitochondrial DNA probes. Specifically, these probes are PCR products amplified from mouse genomic DNA (Kalinowski et al., 1992). The amount of virtual crosslinks in each sample are determined as previously described (Cullinane et al., 2000).

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

### REFERENCES

Ashby, J. and Lefevre, P. A. (1982) Formaldehyde generators: relationship between stability, lipophilicity and carcinogenic potency Carcinogenesis Vol 3, No 11 pp 1273-1276, 1982).

Aviram A, Zimrah Y, Shaklai M, Nudelman A and Rephaeli A (1994) Comparison between the effect of BA and its prodrug pivaloyloyxmethyl butyrate on histones hyperactylation in an HL-60 cell line. Int J Cancer 56, 906-909.

Bishop J F (1999) “Cancer Facts. A Concise Oncology Text”, Harwood, Australia. Bundgaard H and Nielsen N M (1987) Esters of N,N-disubstituted 2-hydroxyacetamides as novel highly biolabile prodrug type for carboxylic acid agents. J Med Chem 30, 451-454.

Chabner B A and Myers C E (1993) In Cancer: Principles and Practice of Oncology (V T DeVita, S Hellman and S A Rosenberg, eds.) pp. 376-381.

Cullinane C, Cutts S M, Panousis C and Phillips D R (2000) Interstrand cross-linking by adriamycin in nuclear and mitochondrial DNA of MCF-7 cells. Nucleic Acids Res 28, 1019-1025.

Cutts, S. M., Rephaeli, A., Nudelman, A., HmeInitsky, I., and Phillips, D. R. (2001) Molecular Basis for the Synergistic Interaction of Adriamycin with the Formaldehyde-Releasing Prodrug AN-9, Cancer Research, in press.

Darzynkiewicz Z, Bolund L and Ringertz N R (1969) Actinomycin binding of normal and phytohaemagglutinin stimulated lymphocytes. Exp Cell Res 55, 120-122.

DeVita V T, Hellman S and Rosenberg S A (1993). Cancer Principles and Practice in Oncology. Vols 1, 2. JB Lippincott: Philadelphia, pp 376-381.

Dicker E, and Cederbaum A I (1984). Inhibition of the oxidation of acetaldehyde and formaldehyde by hepatocytes and mitochondria by crotonaldehyde. Arch Biochem Biophys 234,187-196.

Gewirtz D A (1999) A critical evaluation of the mechanisms of action proposed for the antitumour effects of the anthracycline antibiotics adriamycin and daunorubicin. Biochem Pharmacol 57, 727-741.

Goodman and Gilman's The Pharmacological Basis for Therapeutics (7th ed.).

Kalinowski, D. P., Illenye, S., and Van Houten, B (1992) Analysis of DNA damage and repair in murine leukemia L1210 cells using a quantitative polymerase chain reaction assay. Nucleic Acids Research, 20:3485-3494.

Kasukabe T, Rephaeli A and Honma Y (1997) An anti-cancer derivative of butyric acid (pivalyloxymethyl-butyrate) and daunorubicin cooperatively prolong survival of mice inoculated with monocytic leukemia cells. Br J Cancer 75, 850-854.

Kato S, Burke P J, Fenick D J, Taatjes D J, Bierbaum V M and Koch T H (2000) Mass spectrometric measurement of formaldehyde generated in breast cancer cells upon treatment with anthracycline antitumour drugs. Chem Res Toxicol 13, 509-516.

Keung W M, and Vallee B L (1993) Daidzin: a potent, selective inhibitor of human mitochondrial aldehyde dehydrogenase. Proc Natl Acad Sci USA 90, 1247-¹²⁵I. Kopf-Maier P and Kolon B (1992) An organoid culture assay (OCA) for determining the drug sensitivity of human tumour. Int J Cancer 51, 99-107.

Kopf-Maier P, Kolon B. (1992) An organoid culture assay (OCA) for determining the drug sensitivity of human tumours. Int J Cancer. 51, 99-107.

Langer, Science, 249: 1527, (1990)

March, J. “Advanced Organic Chemistry: Reactions, Mechanisms and Structure” 4^{th} Edition, pp 352-357, John Wiley & Sons, New York, 1992.

Myers C E, Mimnaugh E G, Yeh G C and Sinha B K (1988) in Anthracyclines and anthracenedione-based anticancer agents (J W Lown, ed.) pp. 527-570, Elsevier, Amsterdam.

Nudelman A, Shaklai M, Aviram A, Rabizadeh E, Zimra Y, Ruse M and Rephaeli A (1992) Novel anticancer prodrugs of butyric acid. J Med Chem 35, 687-694.

Parker, B. S., Cullinane, C., and Phillips, D. R. (1999) Formation of DNA adducts by formaldehyde-activated mitoxantrone. Nucleic Acids Research, 27:2918-2923.

Perrin L C, Cullinane C, Kimura K-I and Phillips D R (1999) Barminomycin forms GC-specific adducts and virtual interstrand crosslinks with DNA. Nucleic Acids Res 27, 1781-1787.

Phillips D R and Cullinane C (1999) “Adriamycin” in Encyclopedia of Molecular Biology T E Creighton (ed), John Wiley and Sons, New York, pp 68-72.

Prasad K N (1979) Effect of sodium butyrate in combination with X-irradiation, chemotherapeutic and cyclic AMP stimulating agents on neuroblastoma cells in culture. Experientia 35, 906-908.

Prasad K N (1980) Butyric acid: A small fatty acid with diverse biological functions. Life Sci 27, 1351-1358.

Pratt W B, Ruddon R W, Ensminger W D and Maybaum J (1994) In The Anticancer Drugs Oxford University Press, New York, pp 155-165.

Remington's Pharmaceutical Sciences, 15th ed. Easton: Mack Publishing Co., 1405-1412,1461-1487 (1975).

Sweatman T W and Israel M (1997) In Cancer Therapeutics, Experimental and Clinical Agents (B A Teicher, ed.) Humana Press, Totowa, N. J., pp 113-135.

The National Formulary XIV., 14^{th} ed. Washington: American Pharmaceutical Association (1975).

Vidali G, Boffa L C, Bradbury E M and Allfrey F G (1978) Butyrate suppression of histone deacetylation leads to accumulation of poly-acetylated forms of H3 and H4 and increased Dnase-1 sensitivity of associated DNA sequences. Proc Natl Acad Sci (USA) 75, 2239-2243.

Weiss R B (1992) Will we ever find a better doxorubicin? Semin Oncol 19, 670-686.

Zeman S M, Phillips D R and Crothers D M (1998) Characterisation of covalent adriamycin-DNA adducts. Proc Natl Acad Sci (USA) 35, 11561-11565.

The claims defining the invention are as follows:

1. A method of treating cancer, comprising the step of administration to a subject in need thereof an effective amount of an aldehyde-releasing compound selected from:
(a) an aldehyde releasing compound which upon hydrolysis by esterase releases two or more equivalents of aldehyde,
(b) an aldehyde-releasing compound which upon hydrolysis by esterase releases an inhibitor of an aldebrde detoxifying agent and an aldehyde, and
(c) an aldehyde-releasing compound which is coupled to a cellular or subcellular targeting sequence or a tumour localising component, or an antibody or an antibody-derived fragment specific for a tumour cell marker,
which increases or supplements the intracellular levels of endogenous aldehyde, prior to, together with, or subsequent to the administration of a therapeutically-effective amount of a chemotherapeutic agent, wherein the efficacy of the chemotherapeutic agent is enhanced relative to the efficacy of the chemotherapeutic agent alone.

2. A method of preferentially forming a chemotherapeutic agent-DNA adduct, comprising the step of administering to a subject in need thereof an effective amount of an aldehyde-releasing compound selected from:
(a) an aldehyde-releasing compound which upon hydrolysis by esterase releases two or more equivalents of aldehyde,
(b) an aldehyde-releasing compound which upon hydrolysis by esterase releases an inhibitor of an aldehyle detoxifying agent and an aldehyde, and
(c) an aldehyde-releasing compound which is coupled to a cellular or subcellular targeting sequence or a tumour localising component, organ antibody or an antibody-derived fragment specific for a tumour cell marker,
prior to, together with, or subsequent to the administration of a chemotherapeutic agent, wherein the chemotherapeutic agent more readily forms DNA adducts than when compared to the chemotherapeutic agent alone.

3. A method according to claim 1 or claim 2, wherein the chemotherapeutic agent is an anthracycline selected from the group consisting of Adriamycin, daunoucfin, idarubicin and epirubicin, or an anthracrenedione.

4. A method according to claim 3, wherein anthracenedione is mitoxantrone.

5. A method according to claim 3, wherein the anthracycline is Adriamycin.

6. A method according to any one of claims 1 to 5S wherein the aldehyde-releasing compound contains at least one —CHR— unit with groups immediately adjacent to this unit that decompose or hydrolyse in situ, wherein R is hydrogen or a C1-4 alkyl, alkenyl or alkynyl.

7. A method according to any one of claims 1 to 6, wherein the aldehyde-releasing compound releases formaldehyde.

8. A method according to any one of claims 1 to 7, wherein the subject is a mammal.

9. A method according to any one of claims 1 to 7, wherein the subject is human.

10. A method according to any one of claims 1 to 9, wherein the cancer is selected from the group consisting of prostate, colon, rectal, breast, both the MX-1 and the MCF lines, pancreatic, neuroblastoma, rhabdomysarcoma, bone, lung, murine, melanoma, leukemia, pancreatic, melanoma, ovarian, brain, head & neck, kidney, mesothelioma, sarcoma, Kaposi's sarcoma, stomach, uterine and lymphoma.

11. A method according to any one of claims 1 to 10, wherein the aldehyde-releasing compound and/or chemotherapeutic agents are administered orally, topically, or parenterally in dosage unit formulations with pharmaceutically acceptable carriers, adjuvants, and/or vehicles.

12. A method according to claim 11, wherein the parenteral administration is selected from the group consisting of subcutaneous injection, intravenous injection, intramuscular injection, intrathecal injection, intracranial injection and infusion techniques.

13. A method according to any one of claims 1 to 12, wherein the compound releases aldehyde mainly in tumour tissue.

14. A method according to any one claims 1 to 13, wherein the aldehyde-releasing compound is of type (a) defined in claim 1 or claim 2.

15. A method according to claim 14, wherein the aldehyde-releasing compound is of formula (II):
Z-(L-M¹-CHR-M²)ₓ (II)
wherein:
x is an integer of 2 or more;
Z is a direct bond or a linking group of valency x;
L is either a direct bond or a spacer group;
R is Pr or C₁₋₄ alkyl, alkenyl or alkynyl;
M¹ is a decomposable or hydrolyzable group; and
M² is a second decomposable or hydrolysable group.

16. A method according to claim 14, wherein, in formula II, Z in N, P(═O), PO, O, S, an optionally substituted C1-20 alkylene, alkenylene or alkynylene chain, which may optionally be interspersed with one or more aryl or heteroaryl groups (which may also be optionally substituted) and/or one or more O, S or N atoms; or Z is an optionally substituted heterogenous cyclic group, an optionally substituted aryl group or optionally substituted heteroaryl group.

17. A method according to claim 15 or claim 16, wherein, in formula II, L is NW, Nalkyl, P(═O)Oalkyl, O, S, an optionally substituted C1-20 alkylene, alkenylene or alkynylene chain, which may optionally be interspersed with one or more aryl or heteroaryl groups (which may also be optionally substituted) and/or one or more O, S or N atoms; or L is an optionally substituted heterogenous cyclic: group, an optionally substituted aryl group or optionally substituted heteroaryl group.

18. A method as claimed in claim 15, wherein, in formula II, x is 2, and Z and L are each a direct bond, such that M¹ of one of the bracketed groups (“chain a”) is directly connected to M¹ of the second of the bracketed groups (“chain b”).

19. A method as claimed in claim 18, wherein, in formula II, M^{1a}-M^{1b} is —O—C(═O)—C(═O)—C—O—.

20. A method as claimed in claim 18, wherein in formula II, M^{1a} and M^{1b} are together O—C(═O)—O.

21. A method as claimed in any one of claims 15 to 20, wherein, in formula II, each M² in the compound is independently —OR¹; —NHR²; —NR³R⁴; —SR⁵; —OAcyl; —SAcyl, a phosphorous acid radical, a phosphoramide radical, halo or hydrogen;
in which:
R¹, R², and R⁵ are each independently hydrogen, optionally substituted alkyl, optionally substituted alkenyl, optionally substituted alkynyl, optionally substituted aryl, optionally substituted heteroaryl group, optionally substituted aralkyl, optionally substituted aralkenyl or optionally substituted aralkynyl group, and
R³ and R⁴ each independently have the same definitions as R¹, R², and R⁵ above, or R³ and R⁴ may together with the nitrogen atom form an optionally substituted heterocyclic group.

22. A method as claimed in claim 15, wherein, in formula II, each M¹ is —C(═O)O—.

23. A method as claimed in claim 15, wherein, in formula II, the compound is of the formula (II):
X′—CH, —OOC-Z′-COO—CH₂—Y′ (III)),
in which X′ and Y′ each have the same definition as M² in the compound of formula (II) as defined in claim 16, and Z′ has the same definition as Z in the compound of formula (II) as defined in claim 16 where Z has a valency of 2.

24. A method as claimed in claim 232, wherein, in formula II, Z′ is an optionally substituted cyclic alkylene, an optionally substituted cyclic alkenylene, an optionally substituted cyclic alkynylene, an optionally Substituted heterocyclic group, an optionally substituted aryl or an optionally substituted heteroaryl.

25. A method as claimed in claim 24, wherein, in formula II, the fragment —OOC-Z′-COO— in the compound of formula (III) is a fragment from a dicarboxy-substituted saturated or unsaturated cyclic diacid, which is an alicyclic, heterocyclic, aromatic or heteroaromatic ring system.

26. A method as claimed in claim 24, wherein, in formula II, Z′ is a direct bond, such that the fragment —OOC-Z′-COO— is derived from oxalic acid.

27. A method as claimed in claim 24, wherein, in formula II, Z′ is alkylene or alkenylene.

28. A method as claimed in claim 16, wherein the aldehyde-releasing compound of formula (II) is coupled to a cellular or subcellular targeting sequence or a tumour-localising component.

29. A method as claimed in claim 28, wherein the cellular or subcellular targeting sequence or a tumour-localising component is a polyclonal or monoclonal antibody.

30. A method as claimed in claim 29, wherein the cellular or subcellular targeting sequence or a tumour-localising component is a monoclonal antibody.

31. A method as claimed in claim 15, wherein, in formula II, at least one M₂ is a radical based on an inhibitor of an aldehyde detoxifying agent.

32. A method according to any one of claims 1 to 13, wherein the aldehyde-releasing compound is of type (b) defined in claim 1.

33. A method according to claim 32, wherein the inhibitor of an aldehyde detoxifying agent is an inhibitor of gamma-glutamyl synthetase or an inhibitor of alcohol and/or aldehyde dehydrogenases.

34. A method according to claim 33, wherein the inhibitor is buthionine sulfoximine or crotonaldehyde, or a derivative of one of these inhibitors.

35. A method according to claim 34, wherein the aldehyde-releasing compound is of the formula (IV)
M³-CHR-M⁴ (IV)
wherein
M³ and M⁴ are each independently a hydrolysable or decomposable group, and
M³ and/or M⁴ and/or R is a radical based on an inhibitor of an aldehyde detoxifying agent.

36. A method according to claim 35, wherein M³ and/or M⁴ is a buthionine sulfoximine radical.

37. A method according to claim 35, wherein R is a 2-butenyl radical such that crotonaldehyde is released on hydrolysis.

38. A method according to claim 35, wherein M⁴ is OR¹; —NHM²; —NR³R⁴; —SR⁵; —OAcyl; —SAcyl, a phosphorous acid radical, a phosphoramide radical, halo or hydrogen;
in which:
R¹, R², and R⁵ are each independently hydrogen, optionally substituted alkyl, optionally substituted alkanyl, optionally substituted alkynyl, optionally substituted aryl, optionally substituted heteroaryl group, optionally substituted aralkyl, optionally substituted aralkenyl or optionally substituted aralkynyl group, and R³ and R⁴ each independently hare the same definitions as R¹, R², and R⁵ above, or R³ and R⁴ may together with the nitrogen atom form an optionally substituted heterocyclic group.

39. A method according to any one of claims 35 to 30, in which the aldehyde-releasing compound of formula (IV) is coupled to a cellular or subcellular targeting sequence or a tumour-localising component.

40. A method as claimed in claim 39, wherein the cellular or subcellular targeting sequence or a tumour-localising component is a polyclonal or monoclonal antibody.

41. A method as claimed in claim 39, wherein the cellular or subcellular targeting sequence or a tumour-localising component is a monoclonal antibody.

42. A method according to any one of claims 1 to 13, wherein the aldehyde-releasing compound is of type (c) defined in claim 1 or claim 2.

43. A method according to claim 42, wherein the antibody is either polyclonal or monoclonal antibody.

44. A method according to claim 42, wherein the antibody is a monoclonal antibody.

45. A pharmaceutical composition comprising:
(i) a chemotherapeutic agent which is a primary or secondary amine, and
(ii) an aldehyde-releasing compound selected from;
(a) an aldehyde-releasing compound which upon hydrolysis by esterase releases two or more equivalents of aldehyde,
(b) an aldehyde-releasing compound which upon, hydrolysis by esterase releases an inhibitor of an aldehyde detoxifying agent and an aldehyde, and
(c) an aldehyde-releasing compound which is coupled to a cellular or subcellular targeting sequence or a tumour localising component, or an antibody or an antibody-derived fragment specific for a tumour cell marker,
together with a pharmaceutically-acceptable carrier.

46. A pharmaceutical composition comprising one or more aldehyde-releasing compounds selected from;
(a) an aldehyde-releasing compound which upon hydrolysis by esterase releases two or more equivalents of aldehyde
(b) an aldehyde-releasing compound which upon hydrolysis by esterase releases an inhibitor of an aldehyde detoxifying agent and an aldehyde, and
(c) an aldehyde-releasing compound which is coupled to a cellular or subcellular targeting sequence or a tumour localising component, or an antibody or an antibody-derived fragment specific for a tumour cell marker,
together with a pharmaceutically-acceptable carrier.

47. A pharmaceutical composition as claimed in claim 45 or claim 46, wherein the aldehyde-releasing compound is of formula II as defined in any one of claims 15 to 31, or of formula IV as defined in anyone of claims 35 to 41.

48. Use of an aldehyde-releasing compound selected from;
(a) an aldehyde-releasing compound which upon hydrolysis by esterase releases two or more equivalents of aldehyde,
(b) an aldehyde-releasing compound which upon hydrolysis by esterase releases an inhibitor of an aldehyde detoxifying agent and an aldehyde, and
(c) an aldehyde-releasing compound which is coupled to a cellular or subcellular targeting sequence or a tumour localising component, or an antibody or an antibody-derived fragment specific for a tumour cell marker,
in the manufacture of a medicament for the treatment and/or prophylaxis of cancer.

49. Use as claimed in claim 48, wherein the aldehyde-releasing compound is of formula II-as defined in any one of claims 15 to 31, or of formula IV as defined in any one of claims 35 to 41.

50. A method of treating cancer comprising the steps of:
(a) determining the optimum time of administration of a therapeutically effective amount of an aldehyde-releasing compound relative to the administration of a chemotherapeutic agent wherein the optimum time is determined by the number of DNA adducts formed;
(b) determining the amount of aldehyde-releasing compound relative to the amount of chemotherapeutic agent;
(c) from step (a) and (b) determining-the amount and timing of delivery of aldehyde-releasing compound and chemotherapeutic agent and administering to a patient in need thereof.

51. An aldehyde-releasing compound of formula (II):
Z-(L-M′-CHR-M²)ₓ (II)
wherein:
X is an integer of 2 or more;
Z is e; direct bond or a linking group of valency x;
L is either a direct bond or a spacer group;
R is E or C1-4 alkyl, alkenyl or alkynyl;
M¹ is a decomposable or hydrolysable group; and
M² is a second decomposable or hydrolysable group,
coupled to a cellular or subcellular targeting sequence or a tumour-localising component.

52. A compound as claimed in claim 51, wherein the cellular or subcellular targeting sequence or tumour-localising component is a polyclonal or monoclonal antibody.

53. A compound as claimed in claim 51, wherein the cellular or subcellular targeting sequence or a tumour-localising component is a monoclonal antibody.

54. An aldehyde-releasing compound as claimed in claim 51, wherein the compound coupled to a cellular or subcellular targeting sequence or a tumour-localising component is of the formula (III):
X′-CH₂—OOC-Z′-COO—CH₂-Y′ (III),
in which X′ and Y′ each have the same definition as be in the compound of formula (II) as defined in claim 51, and Z′ has the same definition as in the compound of formula (II) as defined in claim 51 where Z has a valency of 2.

55. An aldehyde-releasing compound as claimed as claimed in claim 54, wherein the fragment —OOC-Z′—COO— in the compound of formula (III) is a fragment from a dicarboxy-substituted saturated or unsaturated cyclic diacid, which is an alicyclic, heterocyclic, aromatic or heteroaromatic ring system.

56. An aldehyde-releasing compound as claimed in claim 54, wherein Z′ is a direct bond, such that the fragment —OOC-Z′-COO— is derived from oxalic acid.

57. An aldehyde-releasing compound as claimed in claim 54 wherein Z′ is alkylene or alkenylene.

58. A compound which includes the aldehyde-releasing compound of formula (XI):
Z-(L-M¹-CHR-M²)ₓ (II)
wherein:
x is an integer of 2 or more;
Z is a direct bond or a linking group of valency X;
L is either a direct bond or a spacer group;
R is E or C₁₋₄ alkyl, alkenyl or alkynyl;
M¹ is a decomposable or hydrolysable group;
wherein M² is a radical based on an inhibitor of an aldehyde detoxifying agent.

59. An aldehyde-releasing compound including a radical based on an inhibitor of an aldehyde detoxifying agent, which aldehyde releasing compound releases said inhibitor and an aldehyde on hydrolysis or decomposition in situ.

60. An aldehyde-releasing compound according to claim 58, wherein the inhibitor of an aldehyde detoxifying agent is an inhibitor of gamma-glutamyl synthetase or an inhibitor of alcohol and/or aldehyde dehydrogenases.

61. An aldehyde-releasing compound according to claim 58, wherein the inhibitor is buthionine sulfoximine or crotonaldehyde, or a derivative of one of these inhibitors.

62. An aldehyde-releasing compound of the formula (IV):
M³-CHR-M⁴ (IV)
wherein
M³ and M⁴ are each independently a hydrolysable or decomposable group, and
M³ and/or M⁴ and/or R is a radical based on an inhibitor of an aldehyde detoxifying agent, coupled to a cellular or subcellular targeting sequence or a tumour-localising component.

63. A compound as claimed in claim 62, wherein the cellular or subcellular targeting sequence or a tumour-localising component is a polyclonal or monoclonal antibody.

64. A compound as claimed in claim 62, wherein the cellular or subcellular targeting sequence or a tumour-localising component is a monoclonal antibody.

65. An aldehyde-releasing compound according to claim 62, wherein M³ and/or M⁴ is a buthionine sulfoximine radical.

66. An aldehyde-releasing compound according to claim 62, wherein R is a 2-butenyl radical such that crotonaldehyde is released on hydrolysis.

67. A method of treating anthracycline or anthracyclinedione-resistant cancer cells, comprising the step of administering to a subject in need thereof an effective amount of an aldehyde-releasing compound which, upon hydrolysis by esterases, releases one or more equivalents of aldehyde,
prior to, together with, or subsequent to the administration of a therapeutically-effective amount of an anthEraLcycline or anthracyclinedione chemotherapeutic agent, wherein the efficiency of the anthracycline or anthracyclinedione chemotherapeutic agent is enhanced relative to the efficiency of the anthracycline or anthracyolinedione chemotherapeutic agent alone.

68. A method according to claim 67, wherein the aldehyde-releasing compound is selected from:
(a) an aldehyde-releasing compound which upon hydrolysis by esterase releases two or more equivalents of aldehyde;
(b) aldehyde-releasing compound which upon hydrolysis by esterase releases an inhibitor of an aldehyde detoxifying agent and an aldehyde, and antibody-derived fragment specific for a tumour cell marker.
